SEQUENCE LISTING (2 sequences)

SEQ ID NO: 1 - protein, 179 aa, Homo sapiens
MAALQKSVSSFLMGTLATSCLLLLALLVQGGAAAPISSHCRLDKSNFQQPYITNRTFMLAKEASLADNNTDVRLIGEKLFHGVSMSERCYLMKQVLNFTLEEVLFPQSDRFQPYMQEVVPFLARLSNRLSTCHIEGDDLHIQRNVQKLKDTVKKLGESGEIKAIGELDLLFMSLRNACI

SEQ ID NO: 2 - protein, 308 aa, synthetic construct
  source 1..308: note = amino acid residues 1-146 represent IL-22, amino residues 147-162 represent the linker, and residues 163-308 represent another IL-22.
APISSHCRLDKSNFQQPYITNRTFMLAKEASLADNNTDVRLIGEKLFHGVSMSERCYLMKQVLNFTLEEVLFPQSDRFQPYMQEVVPFLARLSNRLSTCHIEGDDLHIQRNVQKLKDTVKKLGESGEIKAIGELDLLFMSLRNACIGSGGGSGGGGSGGGGSAPISSHCRLDKSNFQQPYITNRTFMLAKEASLADNNTDVRLIGEKLFHGVSMSERCYLMKQVLNFTLEEVLFPQSDRFQPYMQEVVPFLARLSNRLSTCHIEGDDLHIQRNVQKLKDTVKKLGESGEIKAIGELDLLFMSLRNACI

(19) United States
(12) Patent Application Publication
(10) Pub. No.: US 2024/0277809 A1
(43) Pub. Date: Aug. 22, 2024

(54) F-652 (RECOMBINANT HUMAN INTERLEUKIN-22) FOR TREATMENT OF SCHIZOPHRENIA AND PSYCHOSIS

(71) Applicant: Adonis Sfera, Redlands, CA (US)

(72) Inventor: Adonis Sfera, Redlands, CA (US)

(21) Appl. No.: 18/443,238

(22) Filed: Feb. 15, 2024

Related U.S. Application Data
(60) Provisional application No. 63/485,422, filed on Feb. 16, 2023.
(60) Provisional application No. 63/504,203, filed on May 25, 2023.

(51) Int. Cl. A61K 38/20; C07K 14/54

(52) U.S. Cl. CPC A61K 38/20; C07K 14/54

(57) ABSTRACT

Microbial translocation (MT) is implicated in schizophrenia and other psychotic disorders, by allowing toxins and microorganisms to penetrate the intestinal barrier and enter the systematic circulation, wherein such toxins and microorganisms may be a causal agent for schizophrenia or psychosis. IL-22 has been shown to have protective effects in the gut and preventing lipopolysaccharide (LPS) migration into host tissues and preventing MT. Compositions and formulations of IL-22 and related chemical derivatives are disclosed as treatments for schizophrenia and other psychotic disorders. A form of IL-22 that may be useful is F-652, a dimer of IL-22 having superior pharmaceutic properties than native IL-22.

### FIELD OF THE INVENTION

Formulations of interleukin-22 and related compounds are provided for the treatment of schizophrenia and related psychotic disorders.

### BACKGROUND

Schizophrenia and related psychiatric syndromes are a severe medical problem significantly affecting millions of people, and have been resistant to treatment. As many as half of schizophrenia patients suffer life-long impairment. Social problems such as long-term unemployment, poverty, homelessness, exploitation and victimization are commonly correlated with schizophrenia. Compared to the general population, people with schizophrenia have a higher suicide rate (about 5% overall) and more physical health problems, leading to an average decrease in life expectancy by 20 to 28 years.

Schizophrenia affects approximately 1% of the world's population. The onset of illness is typically between the late teens and mid-thirties. Schizophrenia is a chronic, disabling disease is characterized by abnormal behavior and psychosis, with symptoms categorized into to positive (e.g., hallucinations and delusions) and negative (e.g., social withdrawal and lack of emotion, energy, and motivation) domains. In many people, schizophrenia is a lifelong disorder. Many people with schizophrenia have other mental disorders, especially substance use disorders, depressive disorders, anxiety disorders, and obsessive-compulsive disorder. Moreover, people with schizophrenia are often admitted to hospitals and frequently experience social problems such as long-term unemployment, poverty, homelessness, exploitation, and victimization. The social cost of this disease is high.

The primary treatment for schizophrenia and other psychoses is the use of antipsychotic medications, but many patients do not respond and there are often significant side effects from these drugs and patient compliance is a frequent problem. New treatments are urgently needed for schizophrenia.

### Microbial Translocation and Schizophrenia

Emil Kraepelin believed that “dementia praecox,” the disorder we now call schizophrenia, was the result of brain poisoning with toxins generated in other parts of the body, especially the mouth, intestine, or genitals (1). In this regard, Kraepelin hinted at the microbiome and believed that microbial molecules could drive the pathogenesis of severe psychiatric illness. However, the infectious model of schizophrenia drew limited attention until the launching of Human Microbiome Project and the discovery of innate lymphoid cells (ILCs).

Several recent studies have connected schizophrenia with microbial translocation (MT) from the gastrointestinal (GI) tract into the host systemic circulation, eventually reaching the brain (2-5). MT refers to the migration of intestinal microorganisms or toxins into host tissues by passing through the intestinal barrier and lamina propria to reach mesenteric lymph nodes and the systemic circulation. In the central nervous system (CNS), bacteria or their components can trigger psychosis by several mechanisms, including inflammation, impaired autophagy, premature cellular senescence, and aberrant microglial activation (6-9).

The evidence for a role by MT in schizophrenia includes:
Patients with schizophrenia have a high prevalence of inflammatory bowel diseases (IBD), such as ulcerative colitis and Chron's disease, conditions associated with increased gut barrier permeability and MT (10-12).
Patients with schizophrenia present with elevated bacterial translocation markers, including soluble CD14 (sCD14) and lipopolysaccharide binding protein (LBP), emphasizing microbial migration from the immune tolerant GI tract into the bacteria-intolerant systemic circulation (13-15).
Patients with schizophrenia exhibit increased blood brain barrier (BBB) permeability, enabling bacteria and/or toxins to ingress the CNS (16-19).
The 2011 outbreak of Escherichia coli (E. coli) in Germany has been associated with the new onset psychosis, connecting this pathogen to neuropsychiatric illnesses (20-21). In addition, E. coli has been implicated in urinary tract infection (UTI), conditions often associated with new onset psychosis or schizophrenia exacerbation, linking this bacterium to psychopathology (22-23).

Although there are a number of approved treatments for schizophrenia, an individual patient may require several trials with different antipsychotic drugs before an effective and reasonably tolerated treatment is identified. For many patients, existing treatment options are unable to adequately control their symptoms or may cause intolerable adverse reactions. Accordingly, new treatment options for schizophrenia are urgently needed.

### SUMMARY OF THE INVENTION

Recombinant human interleukin-22 (IL-22), F-652, is expected to alleviate schizophrenic and other psychotic symptoms by blocking microbial translocation (MT) from the gastrointestinal (GI) tract into the host systemic circulation. The migration of intestinal microorganisms or toxins into the systematic circulation caused by MT is blocked by IL-22.

In some aspects, this invention relates to a method of treating schizophrenia or other psychotic disorders, with the administration of a pharmaceutical formulation containing and active agent including IL-22 or a dimer, fusion protein or conjugate thereof to a person suffering from schizophrenia or psychosis.

In some aspects, this invention relates to a method wherein the active agent is F-652.

In some aspects, this invention relates to a method wherein the active agent is administered in an injectable formulation containing at least one excipient selected from a bulking agent, a buffering agent, a antimicrobial preservative, a chelating agent, an antioxidant, a reducing agent, a solvent, and a cosolvent, or a combination thereof. The injection may be administered intravenously or by intramuscular injection.

In some aspects, this invention relates to a method wherein the dose of the active agent is 2 μg/kg of body weight to 200 μg/kg of body weight.

In some aspects, this invention relates to the use of F-652 for the treatment of schizophrenia or for the manufacture of a medicament for the treatment of schizophrenia.

In some aspects, this invention relates to a method of treating schizophrenia including inhibiting microbial translocation in the intestines with the administration of an interleukin-22.

In some aspects, this invention relates to a method of treating schizophrenia including inhibiting microbial translocation in the intestines with the administration of a recombinant interleukin-22 having the sequence of SEQ ID. 001.

In some aspects, this invention provides a pharmaceutical composition for the treatment of schizophrenia, wherein the composition comprises an interleukin-22 agonist. The interleukin-22 agonist may be F-652.

### BRIEF DESCRIPTION OF THE DRAWINGS

FIG. 1. HIV induces apoptotic loss of innate lymphoid cells, type 3 (ILC3), lowering IL22, the guardian of gut barrier (309). This, in turn promotes MT into the systemic circulation. Activated host immunity maintains a state of low-grade inflammation, a pathology found in schizophrenia.

FIG. 2. Innate lymphoid cells are comprised of NKCs and innate lymphoid cells type 1, 2, and 3 (ILC-1, ILC-2, and ILC-3) (309). These cells are activated by various cytokines (top), release other cytokines (bottom), and express transcription factors, including T-bet, GATA-3 and RORγt. Under pathological circumstances, these lymphoid systems may lead to IBD and microbial translocation (MT), contributing to schizophrenia. A subgroup of gut NKCs produce IL-22, maintaining the gut barrier homeostasis (not shown).

FIG. 3. IL-22 modulates the release of hepcidin from the liver. Hepcidin degrades ferroportin in many cell types, preventing iron egress from cells. This mechanism lowers the extracellular iron and its toxicity. Dysfunctional IL-22/ferroportin axis alters intracellular iron, promoting ferroptosis. Body iron is derived from absorption in the gut and from recycling of old erythrocytes by macrophages in the spleen. Exogenous administration of IL-22 (or F-652) may regulate hepcidin, rescuing cells, including the neurons, from cell death by ferroptosis.

FIG. 4. Sources of IL-22 and downstream signaling pathways: IL-22 is mainly secreted by immune cells. JAK1 (Janus kinase 1) and TYK2 (tyrosine kinase 2) molecules are associated with the cytoplasmic region of IL-22 receptor. When the IL-22 receptor is activated (due to a complex formation between its two heterodimeric receptors), JAK1 and TYK2 are phosphorylated, which further phosphorylate STAT (signal transducer and activator of transcription) molecules thus resulting in dimerization and consequent translocation of STATs to the nucleus. Usually STAT3 molecules play key role, but STAT1 and STATS may also be involved. Other than JAK/STATactivation and downstream signaling, IL-22 also activates PI3K/AKT (phosphoinositide 3-kinases/protein kinase B) and MAPK (mitogen-activated protein kinase) pathways through JAK and TYK2 molecules (32).

FIG. 5. IL-22 attaches to both IL-22R1 and IL-10RB receptors, therefore, its agonist, F-652 will exert IL-22 and IL-10 actions. It activates STAT3 and MAPK, effector molecules implicated in schizophrenia and IBD (310).

### DETAILED DESCRIPTION

This disclosure relies on the evidence that microbial translocation (MT) is implicated in schizophrenia and other psychotic syndromes and that recombinant IL-22 derivatives can block MT. MT causes microbes and toxins that normally reside in the gut to “leak”, that is inappropriately penetrate, the intestinal walls into systematic circulation. Evidence shows that such microbes and toxins can be a causal factor in the onset and perhaps maintenance of schizophrenia. Thus, IL-22 and chemical derivatives are expected to have the novel and unexpected effect of treating schizophrenia.

This invention provides methods and compositions for the treatment of schizophrenia or other psychoses with IL-22 and IL-22 derivatives, in particular, the IL-22 dimer F-652.

### Interleukin-22, the “Guardian” of Gut Barrier.

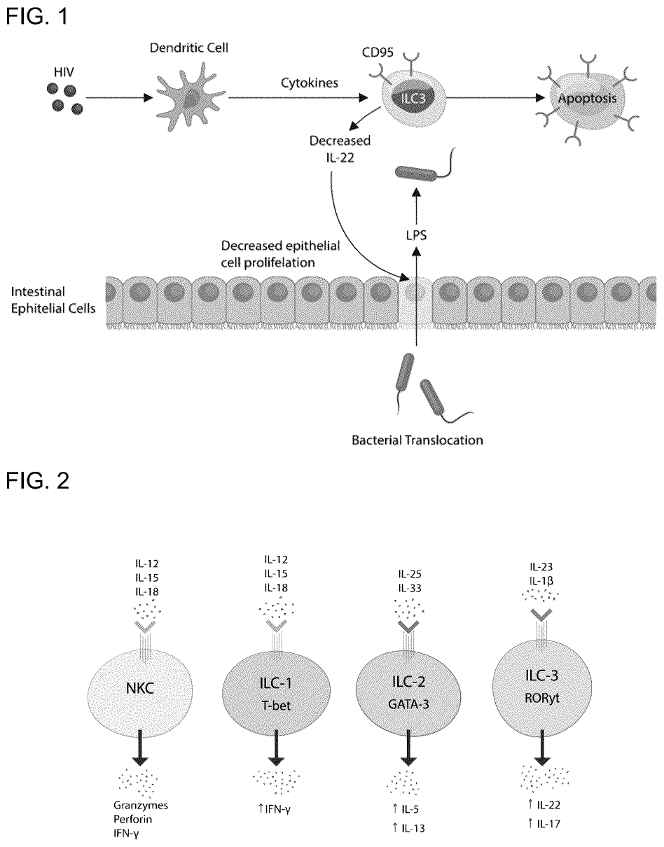

IL-22 has been shown to have protective effects in the gut in preventing lipopolysaccharide (LPS) migration into host tissues and MT. During the human immunodeficiency virus (HIV) epidemic in 1980s, MT drew the attention of researchers and clinicians as this virus depleted interleukin-22 (IL-22), enabling LPS migration into host tissues (22-23)(FIG. 1). Indeed, patients with HIV are more likely to develop new onset psychosis compared to the general population, suggesting that dysfunctional gut barrier may play a role in pathology (24-29) including LPS migration and MT through the intestinal epithelium.

Discovered in 2000, IL-22 is a member of IL-10 family generated by several lymphocyte types, including T helper (Th) 17 cells, yo T cells, natural killer cells (NKCs), and innate lymphoid cells (ILCs)(30). IL-22 controls several intestinal epithelial cells (IEC) functions, including mucus formation, permeability, synthesis of both complement and antimicrobial peptides (AMPs), indicating that this cytokine functions as the master regulator of gut barrier permeability (31).

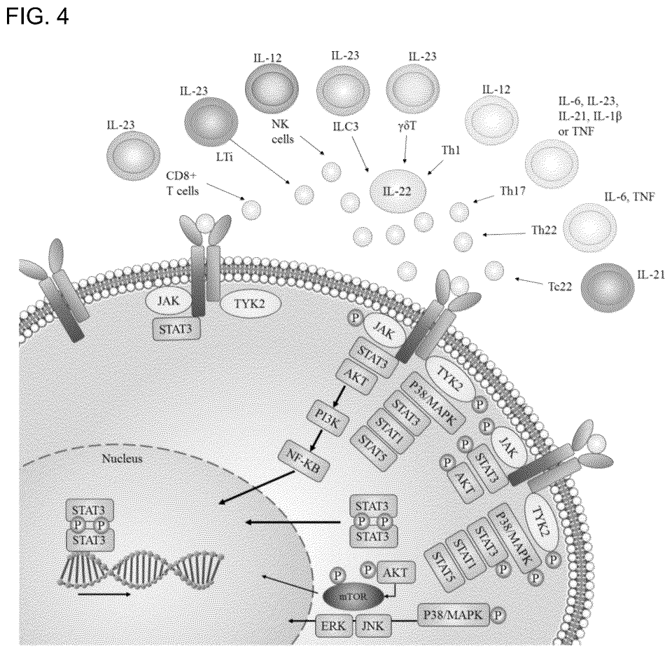

The crosstalk between IL-22 and its receptor (IL-22R), a dimeric protein comprised of IL-22R1 and IL-10R2, activates the JAK/STAT pathway, a key antibacterial and antiviral system, protecting against pathogens (32-33) (FIG. 4). As IL22R contains IL-10R2, it can be cross-activated by IL-10, a cytokine previously connected to schizophrenia (34-36). In addition, several studies have shown that IL-22 possesses neuroprotective properties and its disruption has been associated with schizophrenia (37-39). Moreover, aside from safeguarding neuronal cells, IL-22 protects against COVID-19, influenza, and IBD, indicating that supplementation with this cytokine via F-652 may ameliorate or totally alleviate these pathologies (39-42).

### IL-22 and Innate Lymphoid Cells (ILCs)

Several studies have reported that a subgroup of NKCs produce IL-22, enhancing the gut barrier (43-45) implicated in MT. Indeed, research over the past two decades has connected defective NKCs with both IBD and schizophrenia (46-49). Moreover, in the CNS, NKCs participate in adult neurogenesis, cognition, and cellular senescence, suggesting that under pathological circumstances, these processes may be disrupted (50-53).

NKCs belong to innate lymphoid cells (ILCs), mucosa-anchored non-T, non-B lymphocytes, which also include ILC-1, ILC-2, and ILC-3 (52) (FIG. 2). These systems play a pivotal role in gut barrier homeostasis, nutrient transport, and immune tolerance of both food proteins and gut commensals (52-53). The ILCs in CNS have been implicated in neuropathology, including schizophrenia, major depressive disorder (MDD) and neurodegeneration (55-57).

For example, aberrant activation of NKCs by IL-12, IL-15, and IL-18 was reported in IBD and schizophrenia, linking ILCs to both conditions (58-59). In addition, NKCs-generated interferon-γ (IFN-γ) has been linked to schizophrenia, emphasizing the key role of ILCs in neuropathology. On the other hand, conventional antipsychotic drugs have been shown to suppress IFN-γ, suggesting that at least in part, IL-22 may contribute to the pathogenesis of schizophrenia (60-62). Indeed, dysfunctional IFN-γ was associated with the loss of dopaminergic neurons in Parkinson's disease (PD), connecting ILCs to dopamine pathology (63). Like antipsychotic agents, IL-22 is an IFN-γ inhibitor, suggesting a mechanism through which F-652 may ameliorate the schizophrenia symptoms (64).

Taken together, dysfunctional ILCs, including NKCs, connect IBD with schizophrenia. In contrast, IL-22 (and F-652) likely exhibit antipsychotic properties not only by lowering MT, restoring the integrity of intestinal barrier, but also by suppressing IFN-γ. Moreover, IL-22 also promote protective autophagy, and promotes iron storage in ferritin, lowering the risk of lipid peroxidation, a pathology linked with schizophrenia (see the section on autophagy).

### Psychosis and Gut Microbes

The BBB regulates CNS entry of peripheral molecules, including gut microbes and toxins, by being comprised of specialized endothelial cells kept together by tight junctions. Interestingly, experimental colitis has been shown to increase BBB permeability, indicating a close crosstalk between the gut barrier and BBB that under pathological circumstances may enable intestinal microbes to reach the brain (65).

The link between gut bacteria and schizophrenia has been documented for several decades. For example, antibodies against various E. coli proteins were detected in patients with schizophrenia but not in healthy controls (66). In addition, schizophrenia and colibactin, an E. coli toxin, were demonstrated to trigger premature cellular senescence and thymic dysfunction (67-70). On the other hand, IL-22 drives the regeneration of the thymus, restoring the homeostasis of senescent cells and, according to one study induces rejuvenation (71-73).

Several gut and urinary tract microbes, including E. coli, were associated with schizophrenia, while IL-22 was shown to neutralize this pathogen (74-78). Other gut microbes, including Hafnei alvei, Pseudomonas aeruginosa, Pseudomonas putida, and Klebsiella pneumoniae have been associated with schizophrenia, further substantiating the infectious paradigm in this disorder (79-80).

Taken together, intestinal inflammation can alter the BBB, enabling microbes, including E. coli, to enter the CNS. Antibodies against microbial antigens (which often mimic human proteins) trigger pathology in distant organs, including the brain. IL-22 opposes MT by lowering gut barrier permeability and by enhancing thymic function to strengthen host immune defenses.

### IL-22 Amino Acid Sequence and Production

IL-22, also known as interleukin-10 related T cell-derived inducible factor (IL-TIF), is a glycoprotein secreted by T cells. The expression of IL-22 mRNA was originally demonstrated in IL-9-stimulated T cell lines, IL-9-stimulated mast cell line, as well as concanavalinA activated spleen cells of mouse. The human IL-22 mRNA is mainly expressed in isolated peripheral T cells and are upon stimulation by anti-CD-3 antibody or ConA. IL-22 mRNA is also expressed in the stimulated NK cells. Activated T cells are mainly CD4+ cells, especially Th1 cells via the CD28 pathway.

IL-22 precursor is composed of 179 amino acid residues (the mature peptide is composed of 146 amino acid residues). Dumoutier first reported the IL-22 DNA sequences of cloned mouse and human (307). In addition, Dumoutier owned patents related to IL-22 (U.S. Pat. Nos. 6,359,117 and 6,274,710), whereas Gurney owned the patent related to use of IL-22 in the treatment of human pancreatic disease (U.S. Pat. No. 6,551,799).

IL-22 is mainly expressed in thymus, brain, activated T cells and mast cells, the lectin-stimulated spleen cells (307), interleukin-2/interleukin-12-stimulated NK cells (208) and in a number of organs and tissues, including gut, liver, stomach, kidney, lung, heart, thymus, spleen, upon LPS stimulation (306), in which an increase of the expression of IL-22 in those organs and tissues can be measured.

IL-22 expresses its biological function through the combination of IL-22R1 receptor and IL-10R2 receptor. IL-22R1 is a receptor specific to IL-22 and is expressed in skin, kidney, the digestive system (pancreas, small intestine, liver, large intestine, colon), and the respiratory system (lung, bronchi).

### IL-22 Amino Acid Sequence:

ADPAPISSHC RLDKSNFQQP YITNRTFMLA KEASLADNNT
DVRLIGEKLF HGVSMSERCY LMKQVLNFTL EEVLFPQSDR
FQPYMQEVVP FLARLSNRLS TCHIEGDDLH IQRNVQKLKD
TVKKLGESGE IKAIGELDLL FMSLANACIH HHHHH

See SEQ ID NO:1. Natural human IL-22 is comprised of 179 amino acids and possesses an N-terminal containing 33-amino acid, while Escherichia coli-generated recombinant human IL-22 (rhIL-22) forms a dimer structure in solution and contains two disulfide bonds between amino acids 40-132 and amino acids 89-178 (97-98).

### IL-22 Safety, Pharmacokinetic and Pharmacodynamic Data

F-652 is a recombinant human IL-22 currently in Phase II clinical trials for the treatment of COVID-19, COVID-19 pneumonia, acute pancreatitis, chronic acute liver failure, alcoholic hepatitis, and graft versus host disease (GVHD)(NCT02406651). Several studies have evaluated the safety, pharmacokinetics, pharmacodynamics, and tolerability of F-652, demonstrating that this is a safe compound with favorable pharmacological parameters (81-82).

Aside from functioning as the guardian of intestinal barrier, IL-22 possesses antibacterial and antiviral properties probably by enhancing autophagy (83-85). Impaired autophagy has been documented in both schizophrenia and IBD, while IL-22 (or F-652) enhances autophagy, a property of many antipsychotic drugs, including clozapine (86-94). Moreover, like antipsychotics, F-652 lowers INF-γ and protects intestinal barrier against IBD and MT (60, 64, 95-96).

### Preclinical Data

IL-22 is a member of the IL-10 cytokine family that facilitates tissue healing, especially the epithelial cells. As it is generated by the leukocytes, cells that are in contact with every tissue and organ, IL-22 shows few adverse effects or hypersensitivity in in vitro and in vivo models. Preclinical studies found that IL-22 protects epithelia, promotes regenerative processes, and is well-tolerated (100)(102). Preclinical and translational studies in rodents and cynomolgus monkeys, found anti-inflammatory effects and good tolerability (102). Other preclinical studies outlined similar findings, clinically predictable, manageable, and reversible adverse effects (100). In term of epithelial protection, others found that this compound repairs the barrier function (103).

### IL-22 is a Driver of Autophagy, Including Mitophagy and Ferritinophagy

Autophagy or “self-eating” in Greek, is a physiological mechanism in which defective organelles or misfolded proteins are broken down in lysosomes to avert cell death by accumulation of molecular debris (104). Autophagy is activated by the aggregation of defective proteins in the endoplasmic reticulum (ER), a calcium (Ca2+)-storing intracellular organelle where cell proteins and lipids are synthesized (105). ER stress, caused by excessive pilling up of faulty proteins, activates the unfolded protein response (UPR), a defense mechanism that activates autophagy, restoring homeostasis (106). ER stress sensors, double-stranded RNA-activated protein kinase (PKR)-like ER kinase (PERK), transcription factor 6 (ATF6), and inositol-requiring enzyme 1 (IRE1), detect the misfolded protein load, initiating autophagic degradation (107-109). However, overactivation of UPR can trigger inflammation, leading to aberrant autophagic digestion of healthy mitochondria (mitophagy) and the iron-storing molecule, ferritin (ferritinophagy), pathologies associated with schizophrenia (110-112). Indeed, excessive autophagy of mitochondria and ferritin may upregulate intracellular iron, leading to cell loss by ferroptosis, a non-apoptotic form of cellular and neuronal demise found in schizophrenia (113). In contrast, IL-22 protects the mitochondrion and promotes protective autophagy as well as iron storage in ferritin, opposing schizophrenia (114-117). Since mitochondria are rich in iron, abnormal mitophagy may also trigger iron dysmetabolism, promoting ER stress-mediated neuropathology (112) (118-121). In contrast, IL-22 as well as ER stress-lowering drugs may represent the avantgarde of a new generation of neuropsychiatric treatments (122-123). For example, the UPR pathway is a novel target for major depressive disorder (MDD) and several antidepressant drugs currently utilized in the clinic were demonstrated to lower ER stress, further linking dysfunctional autophagy to neuropsychiatric disorders (124). Moreover, as UPR was found to convert psychological into cellular stress, ER accumulation of molecular waste, likely plays a key role in neuropathology (125-126). Furthermore, chlorpromazine was demonstrated to selectively enhance autophagy, eliminating malignant cells, while sparing the healthy ones (127). Like chlorpromazine, IL-22 activates protective autophagy, facilitating autophagic clearance of malignant, infected or damaged cells (128). In the GI tract, autophagy mediates the crosstalk between gut microbiota and host immunity, while impaired autophagy can disrupt the gut barrier, a characteristic of IBD (129-130). Recent studies found a bidirectional relationship between autophagy and IL-22 as autophagy upregulates this cytokine and in return, IL-22 activates autophagy, protecting the gut barrier (131-132). Recent studies reported an interaction between autophagy and oxidative stress as antioxidants may suppress autophagic implementation (133-134). In this regard, IL-22 not only alleviates ER stress but may also exert antioxidant properties as it was shown to lower doxorubicin-induced oxidative stress, linking it further to ER homeostasis (135-136).

The ER is tethered to the mitochondrion through a distinct sub-compartment, the mitochondria-associated membranes (MAMs), an area linked to both autophagy and schizophrenia (137). For example, disrupted-in-schizophrenia 1 (DISC1) gene, a major risk factor for schizophrenia, is highly expressed in MAMs, connecting these membranes to the neuropsychiatric pathology (138-139). In the GI tract, MAM pathology has been implicated in IBD-mediated gut barrier dysfunction (140-141).

### IL-22 and Iron Metabolism

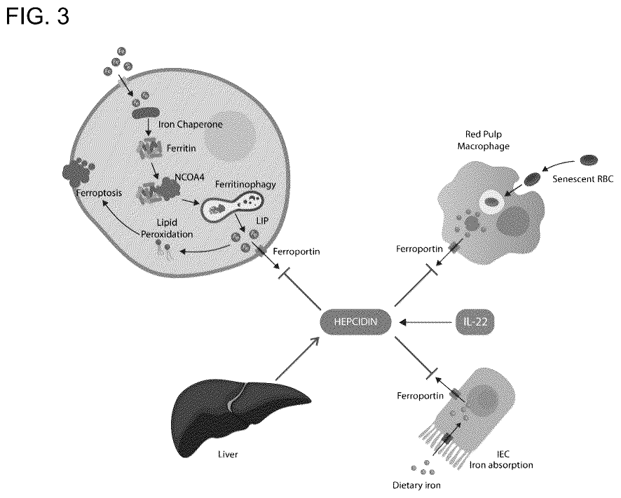

Several studies have demonstrated that in schizophrenia, iron accumulates in several brain regions, while unmedicated, first episode patients, exhibited decreased ferritin, suggesting that excessive ferritinophagy may drive this pathogenesis (112)(142). In addition, lipid peroxidation, an activator of ferritinophagy, was also found elevated in schizophrenia, implicating ferroptosis in this pathology (113)(143). IL-22 induces hepcidin, inhibiting ferroportin, the only known iron-exiting channel, thus, sequestrating the iron in ferritin (144-145) (FIG. 3). This is significant as ferroportin levels were found upregulated in schizophrenia, suggesting that lowering this molecule may be therapeutic (146). Since extracellular iron can be extremely toxic for biological molecules, IL-22-mediated iron-ferritin binding is protective (147-148).

Nutritional immunity is a physiological process of intracellular iron sequestration to deny it to the invading pathogens. Among other effectors, this process is regulated by IL-22 which blocks iron egress from cells via ferroportin by upregulating hepcidin (149-150). In schizophrenia, neuroimaging studies found iron retention in the putamen (a round structure located at the base of the forebrain), suggesting involvement of nutritional immunity, iron sequestration to withhold it from the gut microbes that found their way into the CNS (151). Indeed, anti-LPS and anti-Saccharomyces cerevisiae antibodies were documented in patients with schizophrenia, indicating that gut microbes and their molecules could reach the brain (152-153). A new visualizing technique, gray-level co-occurrence matrix (GLCM), is a computational method for visualizing textural patterns that can be applied in almost any area of microscopy, including identification of Gram-negative bacteria in tissues (154). Along this line, a recent study found that GLCM measures psychological stress-induced chromatin reorganization, suggesting that it may also visualize schizophrenia-related brain LPS and/or microbes (155-156). In addition, as IL-22, the guardian of gut barrier, is lowered by psychological stress, GLCM may be able to quantify microbial translocation by IECs chromatin reorganization (157-158).

D-amino acids are bacterial molecules demonstrated to play a major role in gut microbes' signaling with human IECs (130). At the host-microbe interface, human D-amino acid oxidase (DAO) recognizes bacterial D-amino acids and converts D-tryptophan into aryl hydrocarbon receptor (AhR) ligands that have beneficial effects on the gut barrier (159). AhR and D-amino acids are novel schizophrenia targets involved in the modulation of both neurotransmission and synaptic plasticity (160). For example, D-serine binding to N-methyl-D-aspartate receptor (NMDAR), was found therapeutic in refractory schizophrenia, suggesting that D-amino acids may comprise a signaling system similar to short chain fatty acids (SCFAs) which exert trophic CNS properties (161-162). Indeed, DAO, which degrades D-amino acids, is believed to be overactive in schizophrenia therefore, DAO inhibitors (DAOI) are currently in clinical trials for this disorder (NCT00960219)(163). In addition, DAO was associated with cellular senescence, a schizophrenia-associated phenotype, connecting this illness once more to the microbiome (164-165). IL-22 is generated by several types of AhR-expressing immune cells, including CD4+ lymphocytes, including NKCs, and ILCs, suggesting the existence of an AhR-IL-22 axis that is likely impaired in schizophrenia (166-167).

Taken together, D-amino acids are microbiota-host signaling molecules and AhR ligands. In schizophrenia, overactive DAO may degrade D-amino acids prematurely, disrupting AhR signaling and the subsequent transcription of IL-22.

### IL-22, Microbial Translocation, and the Dopaminergic Hypothesis

IL-22, a member of the IL-10 cytokine family, has been known for its antimicrobial properties, protection of epithelial cells, and tissue repair. In addition, IL-22 possesses anti-apoptotic, pro-proliferative and pro-regenerative characteristics that enhance immune cells survival, proliferation, regeneration of epithelial barriers as well as the thymus (168-170). Interestingly, the central dopamine (DA) system has been shown to promote the integrity of colonic epithelial barrier, linking microbial translocation outside the GI tract to the CNS nigrostriatal pathway (171). This may be significant as dopamine is a ligand of aryl hydrocarbon receptor (AhR), a transcription factor that promotes the expression of IL-22. In view of the antiinflammatory effects of IL-22 on intestinal mucosa, IL-22 is expected to inhibit microbial translocation in the gut (31).

At present, most researchers and clinicians have adopted the dopamine hypothesis (DH) of schizophrenia as the main working model of schizophrenia etiology. The DH postulates that excessive dopamine in the mesolimbic brain pathway activates the postsynaptic receptors, leading to psychosis (172-173). The DH asserts further that the negative and cognitive symptoms of schizophrenia may be due to reduced DA-D1 receptor activation in the prefrontal cortex (174).

The DH is not universally accepted, and it has been critiqued for being built on partially conclusive studies, and for being revised three times since initially published (175). The last “revision” asserts that the interaction of environmental stressors with patient's genetic vulnerabilities upregulates striatal DA, promoting psychosis (175). Moreover, the DH has had even more difficulties accounting for the higher prevalence of schizophrenia in industrialized countries, urban areas, or northern latitude as well as the high comorbidity with IBD, immune abnormalities, and the presence of autoantibodies. For example, increased brain DA usually leads to euphoria and heightened alertness, features less common in schizophrenia, suggesting that DA alone may not adequately explain this syndrome.

### Anosognosia: The Most Common Feature of Schizophrenia

Poor insight or anosognosia (a condition in which a person with a disability is cognitively unaware of their disability due to an underlying physical or psychological condition) is the most frequent symptom of schizophrenia, occurring in 57 to 98 percent of patients, that is not affected by the psychotropic drugs and probably independent of DA (9). However, many patients with schizophrenia exhibit increased peripheral monocyte count, a marker of impaired insight in viral illnesses, including COVID-19 and human immunodeficiency virus (HIV) (176-180). Monocytosis is also a marker of microbial translocation, and along with sCD14 and lipopolysaccharide-binding protein (LBP), precedes the clinical symptoms of schizophrenia (181-182). Blood-derived monocytes are known to infiltrate the CNS and differentiate into phagocytic cells, such as macrophages or microglia, suggesting that increased monocyte count in peripheral blood reflects microglial activation (183). This is significant as excessive phagocytosis of synapses, neurons, and dendritic spines by aberrantly activated microglia were demonstrated in schizophrenia with cognitive and negative symptoms, including anosognosia (184).

Self-awareness and insight have been linked to the immune system distinction between “self” and “non-self”, an idea promoted by Damasio in 2003 (185) and substantiated further by Barnsley in 2011 via the “rubber hand illusion” (186-187). Rubber hand illusion refers to patients who after strokes experience the paralyzed limb as not belonging to them (non-self). When a rubber hand is placed next to the “disowned” one, the brain mistakenly concludes that the fake hand is real, lowering electrical impulses in the patient's real hand and increasing histamine reactivity as if a foreign tissue had been detected, connecting insight to the immunity (187). In light of the “rubber hand illusion”, it is not surprising that monocytosis may represent a biological marker of anosognosia, especially because the AhR controls monocyte differentiation (188).

Dopamine Hypothesis Vs. Microbial Translocation Hypothesis

Aside from its neurotransmitter function, dopamine is an agonist of AhR, a ligand-activated transcription factor known for its role in xenobiotic metabolism and environmental pollutants. In addition, AhR is activated by endogenous agonists, such as tryptophan metabolites and photometabolites, including indole and kynurenine, bilirubin, and 6-Formylindolo[3,2-b]carbazole (FICZ) (189) (Table 1). In addition, AhR binds psychotropic drugs carbidopa and clozapine and is the master regulator of IL-22, suggesting the existence of a DA/AhR/IL-22 axis that likely regulates the brain and gut immunity, and possibly also the insight (190-193). Indeed, IL-22 and AhR oppose not only microbial translocation by lowering the BBB and gut barrier permeability, but also monocyte entry into the CNS, suggesting a role in insight (194-195). Exogenous AhR ligands, environmental pollutants, such as benzotriazole ultraviolet stabilizers and plasticizers, have been associated with both schizophrenia and gut barrier disruption, emphasizing the role of microbial translocation in this disorder (196-197). For example, the plasticizers, bisphenol A and bisphenol S were demonstrated to interfere with the CNS neurotransmission probably leading to schizophrenia or autism spectrum disorders, suggesting that, aside from biological vulnerability, microbial products and environmental factors contribute to the pathogenesis of these illnesses (198-202). In addition, bisphenols interfere with immunity as they bind to the thymic DNA, likely disrupting T cell maturation (203). In this regard, the pollutant/AhR interaction may explain the higher prevalence of schizophrenia in industrialized countries and urban areas compared to developing regions and rural areas that are usually less polluted (204-205). Moreover, the abundance of sunlight-induced vitamin D3 hydroxyderivatives (which also bind to the AhR) may account for the increased prevalence of schizophrenia proportional to the distance from the equator (206). Furthermore, endogenous AhR ligands, tryptophan photometabolites, such as 2,3,7,8-tetrachlorodibenzo-p-dioxin (TCDD), were demonstrated to induce L1 transposon mobilization, a pathology found in schizophrenia (27, 208) (Table 1).

TABLE 1
AhR agonists and antagonists relevant for schizophrenia

| | | | PHARMA- |
|---|---|---|---|
| EXOGENOUS | ENDOGENOUS | DIETARY | COLOGICAL |
| AGONISTS | AGONISTS | ANTAGONISTS | ANTAGONISTS |
| Benzotriazole | Tryptophan | quercetin | CH-223191 |
| UV stabilizer | metabolites and | | |
| | FICZ | | |
| Plasticizers, | Indoles | apigenin | Alpha- |
| Bisphenol | | | naphtoflavone |
| A&S | | | 3-methroxy-4- |
| | | | itriflavone |
| Clozapine | D3 | luteolin | |
| | hydroxyderivatives | | |
| Carbidopa | Dopamine | kaempferol | |

Negative symptoms of schizophrenia, including alogia, avolition, anhedonia, poverty of speech and thought do not respond adequately to antipsychotic drugs and their pathogenesis cannot be explained by the DH (174)(209). Interestingly, the antibiotic minocycline was shown to partially improve the deficit symptoms of schizophrenia, suggesting further that gut microbes may be involved in this pathology (210). Indeed, antibodies against intestinal microbes: Hafnei alvei, Pseudomonas aeruginosa, Morganella morganii, Pseudomonas putida, and Klebsiella pneumoniae were linked to the negative symptoms of schizophrenia (211). Moreover, as these microorganisms are sensitive to minocycline, the amelioration of negative/cognitive symptoms by this antibiotic highlights further that the elimination of etiologic agents is therapeutic (212-216). In addition, minocycline was demonstrated to reduce the peripheral monocyte count, suggesting a potential role in insight improvement (217).

Many conventional antipsychotic drugs exhibit antibacterial properties, suggesting that elimination of translocated microbes could comprise a DA-independent antipsychotic mechanism. For example, phenothiazines and thioxanthenes exert antibacterial activity, while Cryptococcosis and Candidiasis respond well to haloperidol, emphasizing a novel antifungal strategy (218-219). Interestingly, Candida albicans (C. albicans) has been associated with the cognitive impairment in schizophrenia, suggesting that the antipsychotic action of haloperidol could be unrelated to DA (53). Indeed, antifungal drug, ketoconazole was also found beneficial in schizophrenia and schizoaffective disorder, highlighting the possible role of fungi in these pathologies (220-221). C. albicans crosses the BBB by degrading the tight junction protein E-cadherin, a molecule previously implicated in schizophrenia (222-223). Indeed, E-cadherin protects the brain dopaminergic tracts, while its dysfunction was linked to Parkinson's disease (PD) (224). In contrast, IL-22 exhibits anti-Candida properties, suggesting a haloperidol-like action mechanism (225)(Table 2).

In the immune system, dopamine functions as a proinflammatory immunomodulator known for inhibiting the tolerogenic CD8+T regulatory cells (Treg), lymphocytes downregulated in schizophrenia (226-227). Likewise, in AD, Tregs were demonstrated to delay the pathology progression, probably by deactivating microglia, implicating immunity in these disorders (228). Furthermore, DA exerts iron-chelating properties and functions as a siderophore, therefore, several gut microbes, including Enterococcus, generate DA to tap into the host iron pool (229-230). In contrast, IL-22, sequestrates iron in the intracellular compartment, withholding it from pathogens, a property known as nutritional immunity (231).

Several antipsychotic and antidepressant medications inhibit bacterial efflux pumps (proteins that eject antibiotics out of prokaryotic cells), contributing to medication resistance (232). As original antipsychotics were developed from methylene blue, an established antimicrobial, it is not surprising that these drugs exert antibiotic properties by blocking microbial efflux pumps (233). Conversely, while dopamine antagonists exert antibacterial effects, DA promotes bacterial and viral infections (234). The human equivalent of bacterial efflux pumps are P-glycoproteins (P-gp), molecules expressed at the BBB and gut barrier that are upregulated in both schizophrenia and IBD, probably accounting for the impaired barrier function in these conditions (235-236).

Taken together, this data suggests that dopamine blocking may not be the only action mechanism by which antipsychotic drugs exert their action. The antimicrobial properties of these compounds may also account for their beneficial effects as they can clear the translocated microbes.

Many studies have shown that dopamine antagonists do not alter the life-long progression of schizophrenia toward functional disability, manifested primarily by negative and cognitive symptoms (237). Indeed, outcome studies show that sustained recovery occurs in less than 14% of patients within the first five years following a psychotic episode and nearly 20% of people with schizophrenia remain homeless despite treatment, suggesting that dopamine antagonists may not alter the overall evolution of schizophrenia (238-241).

As schizophrenia is characterized by premature cellular senescence, several researchers and clinicians have suggested that senolytic drugs could be useful in this disorder by improving intestinal barrier. For example, preclinical studies have documented that dasatinib and quercetin can improve cognition, linking microbial translocation to this pathology (242-243). In addition, the antihypertensive drug and gut barrier protector, sodium nitroprusside (SNP), was demonstrated to ameliorate the schizophrenia symptoms within minutes after intravenous administration, suggesting that it may arrest the long-term disease progression (244-245). Indeed, SNP-released nitric oxide (NO), a known antimicrobial, has been proposed as a schizophrenia treatment (245). This is bolstered by the finding that knockout mice unable to make neuronal nitric oxide synthase, which makes nitric oxide, showed cognitive impairments and hippocampal protein derangements (246). This is significant as aside from protecting the gut barrier, because SNP also exerts antibacterial actions, suggesting that the progressive cognitive deterioration in schizophrenia may be averted by bactericidal drugs and gut barrier protectors (248). Taken together, this data suggests that IL-22 mediated gut barrier repair may slower or stop the progression of schizophrenia toward functional disability.

As opposed to the gut lumen, outside the GI tract, intestinal microbes are not immunologically tolerated. Indeed, in response to the translocated microbes, the host generates a variety of autoantibodies, including anti-GABA (gamma-aminobutyric acid) and anti-NMDA (N-methyl D aspartate), that were documented in schizophrenia, and cannot be explained by the DH (249-250). However, as the gut microbes express these proteins, upon translocation, these molecules may elicit antibody formation (251). For example, antibodies against E. coli proteins yjjU, livG and ftsE were found in patients with schizophrenia, suggesting that upon translocation, this microbe triggers host antibodies (252). Indeed, older studies found E. coli O-antigen in sera from patients with schizophrenia, further linking this disorder to gut microbes (253). Moreover, GABA-producing Pseudomonas fluorescens, may upon translocation, elicit anti-GABA antibodies, that were also documented in schizophrenia (254-255). Other gut microbes, including Coryneform bacteria, including Corynebacterium glutamicum, produce glutamate, suggesting that anti-NMDA antibodies in schizophrenia may be classical immunoglobulins directed at translocated glutamate producing bacteria (256-257) (Table 2).

Table 2 summarizes a number of features associated with schizophrenia and compares the dopamine hypothesis (DH) to the microbial translocation hypothesis. Table 2 suggests that the MTH provides a superior underlying theory of the etiology of schizophrenia. This conclusion opens new avenues of therapy for this intractable disease.

TABLE 2
DH vs. microbial translocation hypothesis (MTH)

| DH | MTH | References |
|---|---|---|
| Negative symptoms of | Negative symptoms | 7; 42; 44 |
| schizophrenia cannot be | associated with Hafnei | |
| explained by the DH | alvei, Pseudomonas | |
| | aeruginosa, Morganella | |
| | morganii, Pseudomonas | |
| | putida, and Klebsiella | |
| | pneumoniae. | |
| Dopamine receptor | DA antagonists exert | 51; 52; 66 |
| antagonism proves the | antimicrobial properties; | |
| DH. | eliminates translocated | |
| | bacteria. | |
| Dopamine is an AhR | AhR regulates IL-22, binds | 23; 24; 25; 26 |
| ligand. | microbiota-generated | |
| | tryptophan metabolites, | |
| | and plasticizers. | |
| Dopamine blockade does | Poor insight and cognitive | 13; 18; 19; 21; 70; |
| not reduce long-term | symptoms are associated | 91 |
| schizophrenia disabilities. | with monocytosis and | |
| | microbial translocation. | |
| Lower prevalence of | Plasticizers are AhR | 29; 30; 31; 32; 33 |
| schizophrenia in | ligands that are more | |
| developing countries and | represented in | |
| rural areas | industrialized countries | |
| | and urban areas. | |
| Increasing prevalence | Sunlight-driven vitamin D | 39 |
| with distance from the | derivatives are AhR | |
| equator | ligands | |
| Autoantibodies against | Gut microbes express | 85; 86; 87; 88; 90 |
| various proteins | molecules, including GABA | |
| | and NMDA, that can elicit | |
| | antibodies upon | |
| | translocation | |

### IL-22 for IBD

The AhR/IL-22 axis plays an essential role in IBD, and IL-22 is currently in clinical trials for this condition (NCT02847052)(258). In addition, IL-22 promoted by Bacteroides ovatus exhibits promising effects for IBD by producing indole-3-acetic acid, an endogenous AhR ligand (259). Indeed, Bacteroides ovatus ELH-B2 may be the avantgarde of second-generation probiotics for IBD and may also play a role in schizophrenia (260). In addition, IL-22 is indispensable for Bifidobacterium function, a commensal microbe known for its beneficial effect on IBD (261-262). Indeed, several studies found that Bifidobacterium-derived alginic acid, an important protector of intestinal barrier by upregulating several types of T helper (Th) cells, including Th22 (263-264).

### IL-22 and the Thymus

IL-22 signals with its receptor on thymic epithelial cells, inducing thymocyte proliferation (265). For example, after total body irradiation of mice, administration of IL-22 promoted thymic rejuvenation marked by increased number of thymocytes (169)(266).

Thymus-activation-regulated chemokine (TARC), now called circulating C—C motif chemokine ligand 17 (CCL17), was found increased in unmedicated schizophrenic patients, suggesting that thymic pathology may play a role in this disorder (267). CCL17 was also found upregulated in AD, PD, and aging heart, suggesting that it may comprise a marker of premature cellular senescence (267-270). In contrast, preclinical studies found that IL-22 lowers CCL17, at least in the lung, suggesting beneficial properties for cognitive disorders (271).

### Dietary and Pharmacological AhR Antagonists

Excessive AhR activation can disrupt neuronal development, causing growth arrest and apoptosis, a likely mechanism at work in autism and schizophrenia (272-273). Weight gain, one of the most common adverse effects of clozapine, is mediated by Ahr activation and may be preventable by the administration of AhR antagonist CH-223191, emphasizing that dietary and/or pharmacological interventions may have a place in averting the metabolic adverse effects of atypical antipsychotic drugs (192) (Table 2). AhR antagonists have been shown to decrease IL-22 expression, indicating that upstream manipulation of this transcription factor could induce IL-22 secretion (275-277). For example, topical drug tapinarof, an approved psoriasis therapeutic, or cytochrome P4501 inhibitors were demonstrated to upregulate IL-22 (278). Moreover, selective AhR modulators (SAhRMs), such as flavonoids, may upregulate IL-22, while avoiding excessive AhR activation (279). For example, quercetin apigenin, luteolin, kaempferol (KPF) and several common spices, including cloves, dill, thyme, nutmeg, and oregano, exhibit SAhRMs properties, indicating that further research into Ahr can contribute to the development of specific agents that can selectively induce IL-22 expression (280-282) (Table 2). Furthermore, synthetic flavonoids alpha-naphtoflavone, 3-methroxy-4-itriflavone and their derivatives are also endowed with SAhRMs properties, suggesting potential interventions for schizophrenia (283).

### IL-22 Chemical Derivatives

Recombinant human IL-22 is an antibacterial and antiviral molecule that promotes tissue repair and autophagy. Like antipsychotic drugs, F-652 suppresses INF-γ, promoting neuroprotection. In addition, F-652 drives thymic regeneration, opposing premature cellular and immune senescence. In clinical trials, F-652 decreased serum triglycerides, correcting dyslipidemia (often found in patients with schizophrenia), and presented with anti-inflammatory properties, warranting further evaluation for schizophrenia.

The use of IL-22 as a drug was reported for use in the treatment of several human diseases, such as pancreatic disease (U.S. Pat. No. 6,551,799) and viral hepatitis including for promoting hepatocyte survival and proliferation US 20130171100). However, native IL-22 may have drawbacks as a treatment. For example, it was found that treatment of graft vs. host disease (GVHD) with donor T cells engineered to produce IL-22 may cause undesirable effects such as increased skin GVHD (WO2015/070077). Recombinant IL-22, dimers and fusion proteins thereof may not have these shortcomings and have been disclosed in U.S. published application no. 2013/0171100.

F-652 is a dimer of IL-22 that may be useful in this invention. F-652 is a recombinant fusion protein consisting of two human IL-22 molecules linked to the human IgG2 constant region which extends the IgG2-Fc extends the serum half-life. (IgG2-Fc) (81). F-652 has extended and/or improved pharmacokinetic (PK) and pharmacodynamic (PD) properties due to the addition of human Fe fragments at the C-terminal. F-652 is able to protect tissue from damage, inflammation, and infection, and enhances tissue repair by activating STAT3 mediated by IL-22R1 expressed on epithelial cells such as hepatocytes (82). As compared to IL-22 monomer, the IL-22 dimer F-652 is shown to prolong in vivo half-life, improve pharmacokinetic properties of the drug, reduce the injection frequency; especially, it is shown to enhance in vivo activity significantly and thus is likely a superior drug to the native IL-22 monomer. In addition, chemical derivatives such as pegylated IL-22 and covalently modified IL-22 proteins may be of value in this invention. For example, the IL-22 in the present invention can be polymerized by the modification with any activated polyethylene glycol (PEG) with molecular weight of 5,000-100,000 for the purpose of prolonging its half-life time. (US2013/0171100). F-652 is an IL-22 agonist.

IL-22/F-652 share several properties with antipsychotic drugs as summarized in Table 3.

TABLE 3
IL-22/F-652 comparison with antipsychotic drugs

| Antipsychotic drugs | IL 22/F-652 | References |
|---|---|---|
| JAK-STAT activation | JAK-STAT activation | 33; 91 |
| Neuroprotective | Neuroprotective | 37; 92 |
| IFN-γ inhibitor | IFN-γ inhibitor | 60; 64; 93 |
| Activate autophagy | Activate autophagy | 86, 88, 94 |
| Antibacterial/antiviral | Antibacterial/antiviral | 83; 84; 95; 96 |

### Use of F-652 in the Treatment of Psychosis

Like the endogenous IL-22, F-652 is expected to have regenerative properties for intestinal mucosa. In this regard, F-652 is expected to decrease the permeability of gut barrier, averting MT from the GI tract into the host circulation.

Schizophrenia has been associated with increased gut barrier permeability which enables microbial migration outside of the GI tract. As intestinal microorganisms are not immunologically tolerated outside the gut, they trigger inflammatory responses via Nod-like receptor protein-3 (NLRP-3) inflammasome, a pathogenetic mechanism previously associated with schizophrenia (285).

NLRP3-mediated inflammation has been demonstrated in inflammatory bowel disease (IBD), including Crohn's disease (CD) and ulcerative colitis (UC), disorders marked by MT. IBD is highly comorbid with schizophrenia, linking MT to neuropathology (286). For example, 44.7% of patients with schizophrenia present with Crohn's disease and 55.3% with ulcerative colitis (10). This elevated comorbidity between IBD and schizophrenia is hypothesized to have a common denominator: host immune response to microbial antigens.

This hypothesis is based on the following findings:
Gut bacteria generate neurotransmitters, including GABA, DA, NE, 5-HT, glutamate, and histamine, to communicate with host CNS.
Microorganisms, including E. coli and H. pylori, express proteins which resemble human glutamate receptors, suggesting that N-methyl-D-aspartate (NMDA) autoantibodies, documented in schizophrenia, may occur by molecular mimicry (106, 107).
Bacteroides species and Pseudomonas fluorescens express γ-aminobutyric acid (GABA) and GABA receptors, suggesting that autoantibodies against GABA-A receptor α1, detected in schizophrenia, could be conventional antibodies directed at translocated microbes and their antigens (107-109).
Increased plasma immunoglobulins against gut microbes Hafnei alvei, Pseudomonas aeruginosa, Morganella morganii, Pseudomonas putida, and Klebsiella pneumoniae were found in schizophrenic patients with negative symptoms, indicating MT (287).
Urinary tract infections, usually caused by Escherichia coli (E. coli), are known to trigger new onset psychosis or exacerbation of schizophrenia in stable patients, linking neuropathology to microbial pathogens (22).

In this regard, F-652 is expected to regenerate and heal intestinal mucosa, lowering gut permeability and microbial translocation, therefore, ameliorating schizophrenia symptoms.

### F-652 Safety, Pharmacokinetic and Pharmacodynamic Data

The half-life of recombinant human IL-22 protein is less than 2 h in animals, which limits its clinical application. Thus, Tang et al. (81) studied F-652, a recombinant fusion protein consisting of two human recombinant IL-22 molecules linked to the human IgG2 constant region (IgG2-Fc), which had T_{1/2} values ranging from 39.4 to 206 h in cohorts treated with different doses of F-652.

F-652 is currently in Phase II clinical trials for the treatment of COVID-19, COVID-19 pneumonia, acute pancreatitis, chronic acute liver failure, alcoholic hepatitis, and graft versus host disease (GVHD)(NCT02406651). Several studies have evaluated the safety, pharmacokinetics, pharmacodynamics, and tolerability of F-652, demonstrating that this is a safe compound with favorable pharmacological parameters (81-82).

Aside from functioning as the guardian of intestinal barrier, IL-22 possesses antibacterial and antiviral properties probably by enhancing autophagy (83-85). Impaired autophagy has been documented in both schizophrenia and IBD, while IL-22 (or F-652) enhances autophagy, a property of many antipsychotic drugs, including clozapine (85-89). Moreover, like antipsychotics, F-652 lowers INF-γ and protects intestinal barrier against IBD and MT (60)(64)(89-90).

F-562 may be administered intraveneously in 1 ml of water once a week in the dose of 45 μg/kg. Subcutaneous administration is possible, however, the first Phase I study in humans (on healthy male volunteers), found delayed injection site reactions, which presented as erythematous patches (98). A recent study Phase II study on patients with alcohol-associated hepatitis found that doses of 45 μg/kg were associated with a high rate of improvement and reported no significant adverse effects (81).

### F-652/IL-10 Interaction

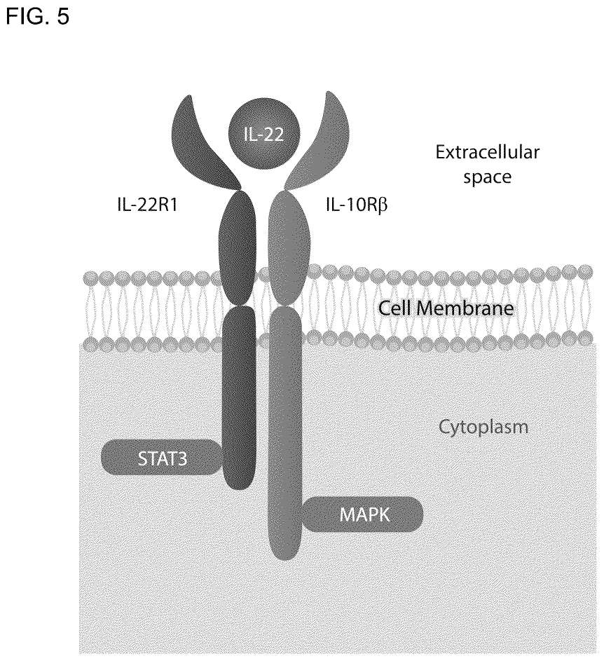

The IL-22 receptor consists of IL-22R1 and IL-10R beta, indicating that IL-10 signaling can be also enhanced by F-652 (FIG. 5). Low IL-10 levels were documented in unmedicated patients with first episode schizophrenia, while IL-22 was found protective against brain autoimmune inflammation (37, 288). In addition, IL-10 signaling has been found to decrease NLRP3 activation and IL-1B production, suggesting beneficial effects for schizophrenia as well as IBD (289). Indeed, IL-1B is considered a driver of both IBD and psychosis (290). Moreover, IL-22 upregulates mitogen-activated protein kinase (MAPK) and signal transducer and activator of transcription 3 (STAT3), molecules altered in IBD and schizophrenia (291).

### F-652 and Brain Neuroepithelia

In the CNS, during the embryonic development, neuroepithelial cells function as neural stem cells, enabling the genesis of new neurons. In the adult brain, neurogenesis continues to take place in several niches, including the dentate gyrus (DG) of the hippocampus, ventricular, and subventricular zones, where new neurons emerge from neural stem/progenitor cells (292). Recently a novel role of IL-22 has emerged: participation in the brain stem/progenitor cell homeostasis and repair (293). It has been established that in schizophrenia there is a paucity of neural stem cells, in the DG which coincides with reduced hippocampal volume and cognitive dysfunction (295). IL-10 was demonstrated to regulate progenitor cell differentiation and modulate the neurogenesis in adult brain, suggesting that F-652 acting via IL-10Rß may not only upregulate neural stem/progenitor cells but also promote their restoration and repair.

Taken together, F-652 is expected to ameliorate schizophrenia by:
Lowering autoimmune neuroinflammation
Lowering MT by decreasing the permeability of gut barrier
Decreasing the activation of NLRP3 in the CNS and GI tract.
Lowering IL-1B
Upregulating STAT3 and MAPK
Upregulating adult neurogenesis and neural cell repair.

### F-652 Preclinical Data

The action of F-652/IL-22 is mediated by binding to IL-22R1 and IL-10RB receptors, followed by activation of STAT3 and MAPK (FIG. 5) (30, 294, 310). Studies of IL-22 transgenic mice with high levels of serum IL-22 (˜600 pg/ml), did not find differences from the wild type and there were no adverse effects noted. Even when mice generated levels close to 6000 pg/ml adverse effects were absent, except lower weight gain, thicker epidermis, and minor skin inflammation (296). Other preclinical studies with IL-22 transgenic mice showed detrimental effects during the embryonic development (preventing lipopolysaccharide-induced preterm labor), but only minor changes after birth (294).

The early studies reported that both Th17 and Th1 secrete IL-22, however, the discovery of Th22 as the IL-22 producing cells without the concomitant release of IFN-γ, IL-4, or IL-17 has attracted much attention to these novel regulatory CD4+ T cells (298). The reason for this is the ability of Th22 to express CCR4, CCR6, CCR10 and several fibroblast growth factors (FGFs) molecules which promote angiogenesis, and accelerate wound healing, including the restoration of biological barriers (299-300).

Aryl-hydrocarbon receptor (AhR), a critical factor for Th22 cell polarization in humans, regulates the expression of IL-22 (301). This is significant as AhR resides primarily in the biological barriers, including gut and BBB and binds several neurotransmitters implicated in schizophrenia (302). For example, dopamine (DA), serotonin (5-HT), and several antipsychotic drugs, including clozapine, are AhR ligands (192). In addition, gut microbiota modulates AhR, which in turn upregulates IL-22, protecting the gut barrier (275).

Studies of F-652 in mice revealed that it protects against epithelial cells from injury, reduces bacterial infection, and inhibits inflammation (304). For example, IL-22 knock out mice exhibited extensive tissue destruction and inflammation (305).

Taken together, the published data suggest that IL-22 agonists, including F-652, are promising drugs for the treatment of epithelial disorders, including biological barrier disorders, and upregulating neural progenitor cells which is accompanied with insignificant adverse effects.

### Manufacture of IL-22 and Derivatives

The active agents of this invention, IL-22 and related compounds, such as F-652, can be produced by methods known in the chemical and biological sciences for producing proteins. F-652 was originally prepared by Generon (Shanghai) Corp. Ltd. as disclosed in US 2013/0171 100 A1. The sequence of F-652 is part of this application as SEQ ID NO: 2.

An IL-22 for use in practicing the method of the present invention can not only be generated using recombinant DNA technology but can also be produced through fusion of heterologous polypeptides. A description of other methods, vectors and host cells for synthesis of an IL-22 for use in practicing the method of the present invention can be found in Gething et al, Nature, 293:620-625; Mantei et al, Nature, 281:40-46; EPI 17,060 and EPI 17,058. Recombinant IL-22, dimers and fusion proteins thereof are produced using methods known to those of skill in the art. For more details, see U.S. published application no. 2013/0171100, the contents of which are hereby incorporated by reference into the present disclosure.

DNA sequences encoding an IL-22 dimer or fusion protein can be entirely synthesized artificially. Alternatively, DNA encoding IL-22 can be obtained by PCR amplification or synthesis and then joined together to form a DNA sequence encoding an IL-22 dimer.

Briefly, suitable host cells are transformed or transfected with an IL-22 expression vector in accordance with known methods and subsequently grown under conditions known to those of skill in the art to promote growth; transfection techniques are described in greater detail in, for example, Molecular Cloning: a Laboratory Manual 3rd edition, J.F. Sambrook and D.W. Russell, ed. Cold Spring Harbor Laboratory Press 2001.

Host cells suitable for expression of IL-22 are also known in the art and include invertebrate cells, such as insect cells, and mammalian cells. Suitable mammalian cells include Chinese Hamster Ovary (CHO), COS cells; in particular, SV40-transformed monkey kidney CV1 cell line (COS-7, ATCC CRL 1651); human embryo kidney cell line 293 (Graham et al, J. Gen Virol, 36:59 (1997)); CHO/-DHFR (Urlaub and Chasin, Proc. Natl. Acad. Sci. USA, 77:4216 (1980)); murine testis trophoblastic cells (TM4, Mather, Biol. Reprod., 23:243-251) (1980)); human lung cells (W138, ATCC CCL 75); human liver cells (Hep G2, HB 8065); murine breast cancer cells (MMT 060562, ATCC CCL51).

A nucleic acid comprising a nucleotide sequence encoding IL-22 can be inserted into a replicable vector for gene cloning or protein expression. Many vectors for protein expression are known in the art. Using these techniques, a nucleic acid sequence encoding IL-22 is inserted into an appropriate vector, which may further include any of the following: one or more signal sequences, an origin of replication, one or more reporter genes, an enhancer element, a promoter, and a transcription termination sequence.

Methods of transfecting eukaryotic cells and transforming prokaryotic cells are also known to those of skill in the art, and may include the use of calcium chloride, calcium phosphate precipitation, lipofectamine or electroporation. One skilled in the art will be able to select a suitable method depending on the host cell selected.

In one embodiment, a recombinant protein containing human IL-22 and produced in CHO cells in serum-free culture is used.

### Dosage

In human volunteers, F-652 was administered via intravenous (IV) infusion at 2.0, 10, 30, and 45 μg/kg and no severe adverse effects were seen (81-82). Dose-dependent transient increase in serum amyloid A and C-reactive protein were reported as well as a decrease of serum triglycerides. The compound was safe and well-tolerated (99).

Preclinical studies and clinical trials phases I and II have shown that F-652 is safe in doses up to 45 μg/kg and associated with reduction in inflammatory markers (measured via Lille and MELD (Model for End-Stage Liver Disease) score (100)(101).

A pharmaceutical composition for use in practicing the method of the present invention includes a safe and effective amount of IL-22 or a dimer, fusion protein or conjugate thereof and may include a pharmaceutically acceptable excipient or carrier. “Safe and effective amount” refers to an amount of a compound sufficient to significantly improve the condition of the patient in need thereof, without causing serious side-effects. In an embodiment, the term “active agent” may mean F-652. In general, a pharmaceutical composition of IL-22 or F-652 comprises 1.0 to 200 μg/kg (body weight) of IL-22 or its dimer per dose; in one embodiment, the pharmaceutical composition comprises 20 to 45 μg/kg of IL-22 or its dimer per dose; in a further embodiment, the pharmaceutical composition comprises 30 μg/kg of IL-22 or its dimer per dose. In a further embodiment, the dosage may be a pharmaceutical composition of 5.0 μg/kg (of F-652) of body weight to 100 μg/kg of body weight, or 25 μg/kg of body weight to 75 μg/kg of body weight, or 45 μg/kg of body weight. In an embodiment, the pharmaceutical composition is a medicament.

The dosage of IL-22 or its dimer may be administered on a schedule that can vary from once per day to once per month. For example, the drug may be given two times per week, once per week, once every other week (14 day intervals), at 21 day intervals, monthly, or once per year.

### Formulations

The IL-22 or dimer, fusion protein or conjugates thereof useful in this invention may be administered to patients suffering from schizophrenia by injection, such as intramuscular (IM) or intravenous (IV) routes of administration.

In an embodiment, F-652 is administered as an infusion in pure water without any excipients. Alternatively, one or more excipients may optionally be used.

Any injectable formulation must be sterile. In some cases, liquid formulations may be shipped to healthcare settings such as hospitals, clinics, or medical offices, or directly to patients as sterile liquid formulations in bottles ready for injection. In other cases, reconstitution may be necessary, typically by adding sterile water optionally containing other ingredients to a powdered material. Powdered materials for reconstitution may include the active ingredient plus one or more excipients. The active ingredient may be lyophilized.

Pharmaceutically acceptable excipients and carriers for injectable formulations of protein drugs are well known to those of skill in the art (284). Considerations for formulations of biologic drugs such as IL-22, or its dimer or chemical derivatives include buffering agents to control the pH, surfactants to stabilize against stress induced aggregation, preservatives to prevent the microbial growth, carbohydrates as bulking agents, salts and sugars to stabilize proteins and maintain the physiological osmolality and tonicity. For proteins, the function of excipients include enhancing solubility and shelf-life stability of the protein, maintaining stable conformation of the active protein and preventing aggregation or degradation of the protein.

For example, useful excipients for formulations in this invention may include one of more of the following agents:

Bulking agents: Sucrose, Lactose, Trehalose, Mannitol, Sorbitol, Glucose, Rafinose, Glycine, Histidine, PVP ((polyvinyl pyrrolidine) K12 to K17

Buffering Agents: acetic acid, and salts (sodium, potassium ammonium), ammonium sulfate, Sodium citrate, Sodium phosphate, Sodium Hydroxide, Tris base, Tris acetate, Tris HCl, sodium bisulfate, sodium sulfite

Antimicrobial Preservatives: Benzalkonium Chloride, Benzethonium Chloride, Benzyl alcohol, Chlorobutanol, m-Cresol, Myristyl gammapicolinium Chloride, Paraben methyl, Paraben propyl, Phenol, 2-Penoxyethanol, Phenyl mercuric nitrate, Thimerosal

Chelating agents: Calcium disodium Ethylenediaminetetra acetic acid (EDTA), Disodium EDTA, Sodium EDTA, Diethylenetriaminepenta acetic acid (DTPA)

Antioxidants and Reducing Agents: Acetone sodium bisulite, Ascorbyl palmitate, Ascorbate (sodium/acid), Bisulite sodium, potassium Metabisulite, sodium Metabisulite, sodium sulfite, Tocopherol alpha

Solvents and Cosolvents: Benzyl benzoate, ethanol, glycerin, N-methyl-2-pyrrolidone, PEG (300-4000), propylene glycol, plant-derived oils: castor, cottonseed, safflower, sesame, olive, soybean, canola

The above lists are representative in terms of functional classes of materials. Other types of functional excipients may be useful besides those listed here. In addition, within each class, only representative materials are shown. Each of these functional classes may have other materials not listed but possessing the indicated pharmaceutic function and that may be useful in formulations of this invention.

### ABBREVIATIONS

AhR aryl hydrocarbon receptor
AMP antimicrobial peptides
BBB Blood brain barrier
CD Crohn's disease
CNS Central nervous system
DA dopamine
DAO D-Amino acid oxidase
DAOI DAO inhibitors
DH dopamine hypothesis
ER Endoplasmic reticulum
FICZ 6-Formylindolo[3,2-b]carbazole
GABA γ-aminobutyric acid
GLCM gray-level co-occurrence matrix
GVHD graft versus host disease
HIV human immunodeficiency virus
IBD inflammatory bowel disease
IEC intestinal epithelial cells
IFN Interferon
IL Interleukin
ILC innate lymphoid cells
JAK Janus kinase
LBP lipopolysaccharide-binding protein
LPS lipopolysaccharide
MAPK mitogen-activated protein kinase
MT microbial translocation
MTH microbial translocation hypothesis
NKC natural killer cells
NLRP-3 Nod-like receptor protein-3
NMDAR N-methyl-D-aspartate receptor
NO nitric oxide
PD pharmacodynamic
PI3K/AKT phosphoinositide 3-kinases/protein kinase B
PK pharmacokinetic
SCFAs short chain fatty acids
SNP sodium nitroprusside
STAT signal transducer and activator of transcription
TYK tyrosine kinase
UC ulcerative colitis

### REFERENCES

1. Noll R. Kraepelin's ‘lost biological psychiatry’? Autointoxication, organotherapy and surgery for dementia praecox. Hist Psychiatry. 2007 September; 18(71 Pt 3):301-20. doi: 10.1177/0957154X07078705.
2. Wang C, Zhang T, He L, Fu J Y, Deng H X, Xue X L, Chen B T. Bacterial Translocation Associates With Aggression in Schizophrenia Inpatients. Front Syst Neurosci. 2021 Sep. 29; 15:704069. doi: 10.3389/fnsys.2021.704069.
3. Severance E G, Gressitt K L, Stallings C R, Origoni A E, Khushalani S, Leweke F M, Dickerson F B, Yolken R H. Discordant patterns of bacterial translocation markers and implications for innate immune imbalances in schizophrenia. Schizophr Res. 2013 August; 148(1-3):130-7. doi: 10.1016/j.schres.2013.05.018.
4. Zhu, F., Ju, Y., Wang, W. et al. Metagenome-wide association of gut microbiome features for schizophrenia. Nat Commun 11, 1612 (2020). https://doi.org/10.1038/s41467-020-15457-9
5. Dickerson F, Severance E, Yolken R. The microbiome, immunity, and schizophrenia and bipolar disorder. Brain Behav Immun. 2017 May; 62:46-52. doi: 10.1016/j.bbi.2016.12.010.
6. Sibony M, Abdullah M, Greenfield L, Raju D, Wu T, Rodrigues D M, Galindo-Mata E, Mascarenhas H, Philpott D J, Silverberg M S, Jones N L. Microbial Disruption of Autophagy Alters Expression of the RISC Component AGO2, a Critical Regulator of the miRNA Silencing Pathway. Inflamm Bowel Dis. 2015 December; 21(12):2778-86. doi: 10.1097/MIB.0000000000000553.
7. Secher T, Samba-Louaka A, Oswald E, Nougayrède J P. Escherichia coli producing colibactin triggers premature and transmissible senescence in mammalian cells. PLOS One. 2013 Oct. 8; 8(10):e77157. doi: 10.1371/journal.pone.0077157.
8. Franchi L, Muñoz-Planillo R, Núñez G. Sensing and reacting to microbes through the inflammasomes. Nat Immunol. 2012 Mar. 19; 13(4):325-32. doi: 10.1038/ni.2231.
9. Fernández-Arjona M D M, Grondona J M, Fernández-Llebrez P, López-Ávalos M D. Microglial activation by microbial neuraminidase through TLR2 and TLR4 receptors. J Neuroinflammation. 2019 Dec. 2; 16(1):245. doi: 10.1186/s12974-019-1643-9. PMID: 31791382
10. Sung K Y, Zhang B, Wang H E, Bai Y M, Tsai S J, Su T P, Chen T J, Hou M C, Lu C L, Wang Y P, Chen M H. Schizophrenia and risk of new-onset inflammatory bowel disease: a nationwide longitudinal study. Aliment Pharmacol Ther. 2022 May; 55(9):1192-1201. doi: 10.1111/apt.16856.
11. Bernstein C N, Hitchon C A, Walld R, Bolton J M, Sareen J, Walker J R, Graff L A, Patten S B, Singer A, Lix L M, El-Gabalawy R, Katz A, Fisk J D, Marrie R A; CIHR Team in Defining the Burden and Managing the Effects of Psychiatric Comorbidity in Chronic Immunoinflammatory Disease. Increased Burden of Psychiatric Disorders in Inflammatory Bowel Disease. Inflamm Bowel Dis. 2019 Jan. 10; 25(2):360-368. doi: 10.1093/ibd/izy235.
12. Marrie R A, Walld R, Bolton J M, Sareen J, Walker J R, Patten S B, Singer A, Lix L M, Hitchon C A, El-Gabalawy R, Katz A, Fisk J D, Bernstein C N; CIHR Team in Defining the Burden and Managing the Effects of Psychiatric Comorbidity in Chronic Immunoinflammatory Disease. Increased incidence of psychiatric disorders in immune-mediated inflammatory disease. J Psychosom Res. 2017 October; 101:17-23. doi: 10.1016/j.jpsychores.2017.07.015
13. Wang C, Zhang T, He L, Fu J Y, Deng H X, Xue X L, Chen B T. Bacterial Translocation Associates With Aggression in Schizophrenia Inpatients. Front Syst Neurosci. 2021 Sep. 29; 15:704069. doi: 10.3389/fnsys.2021.704069.
14. Severance E G, Gressitt K L, Stallings C R, Origoni A E, Khushalani S, Leweke F M, Dickerson F B, Yolken R H. Discordant patterns of bacterial translocation markers and implications for innate immune imbalances in schizophrenia. Schizophr Res. 2013 August; 148(1-3):130-7. doi: 10.1016/j.schres.2013.05.018.
15. Szeligowski T, Yun A L, Lennox B R, Burnet P W J. The Gut Microbiome and Schizophrenia: The Current State of the Field and Clinical Applications. Front Psychiatry. 2020 Mar. 12; 11:156. doi: 10.3389/fpsyt.2020.00156.
16. Najjar S, Pahlajani S, De Sanctis V, Stern J N H, Najjar A, Chong D. Neurovascular Unit Dysfunction and Blood-Brain Barrier Hyperpermeability Contribute to Schizophrenia Neurobiology: A Theoretical Integration of Clinical and Experimental Evidence. Front Psychiatry. 2017 May 23; 8:83. doi: 10.3389/fpsyt.2017.00083.
17. Puvogel, S., Alsema, A., Kracht, L. et al. Single-nucleus RNA sequencing of midbrain blood-brain barrier cells in schizophrenia reveals subtle transcriptional changes with overall preservation of cellular proportions and phenotypes. Mol Psychiatry 27, 4731-4740 (2022). https://doi.org/10.1038/s41380-022-01796-0
18. Greene C, Hanley N, Campbell M. Blood-brain barrier associated tight junction disruption is a hallmark feature of major psychiatric disorders. Transl Psychiatry. 2020 Nov. 2; 10(1):373. doi: 10.1038/s41398-020-01054-3.
19. Cheng Y, Wang T, Zhang T, Yi S, Zhao S, Li N, Yang Y, Zhang F, Xu L, Shan B, Xu X, Xu J. Increased Blood-Brain Barrier Permeability of the Thalamus Correlated With Symptom Severity and Brain Volume Alterations in Patients With Schizophrenia. Biol Psychiatry Cogn Neurosci Neuroimaging. 2022 October; 7(10):1025-1034. doi: 10.1016/j.bpsc.2022.06.006.
20. Kleimann A, Toto S, Eberlein C K, Kielstein J T, Bleich S, Frieling H, Sieberer M. Psychiatric symptoms in patients with Shiga toxin-producing E. coli 0104:H4 induced haemolytic-uraemic syndrome. PLOS One. 2014 Jul. 9; 9(7):e101839. doi: 10.1371/journal.pone.0101839.
21. Wiwanitkit V. Psychosis and E. coli Infection: A Forgotten Issue. Indian J Psychol Med. 2012 October; 34(4):407-8. doi: 10.4103/0253-7176.108241.
22. Graham K L, Carson C M, Ezeoke A, Buckley P F, Miller B J. Urinary tract infections in acute psychosis. J Clin Psychiatry. 2014 April; 75(4):379-85. doi: 10.4088/JCP.13m08469
23. Buchholz U, Bernard H, Werber D, Böhmer M M, Remschmidt C, Wilking H, Delere Y, an der Heiden M, Adlhoch C, Dreesman J, Ehlers J, Ethelberg S, Faber M, Frank C, Fricke G, Greiner M, Höhle M, Ivarsson S, Jark U, Kirchner M, Koch J, Krause G, Luber P, Rosner B, Stark K, Kühne M. German outbreak of Escherichia coli O104:H4 associated with sprouts. N Engl J Med. 2011 Nov. 10; 365(19):1763-70. doi: 10.1056/NEJMoa1106482.
24. Sandler N G, Douek D C. Microbial translocation in HIV infection: causes, consequences and treatment opportunities. Nat Rev Microbiol. 2012 September; 10(9):655-66. doi: 10.1038/nrmicro2848.
25. Kim C J, Nazli A, Rojas O L, Chege D, Alidina Z, Huibner S, Mujib S, Benko E, Kovacs C, Shin L Y, Grin A, Kandel G, Loutfy M, Ostrowski M, Gommerman J L, Kaushic C, Kaul R. A role for mucosal IL-22 production and Th22 cells in HIV-associated mucosal immunopathogenesis. Mucosal Immunol. 2012 November; 5(6):670-80. doi: 10.1038/mi.2012.72.
26. Harris M J, Jeste D V, Gleghorn A, Sewell D D. New-onset psychosis in HIV-infected patients. J Clin Psychiatry. 1991 September; 52(9):369-76. PMID: 1894589.
27. de Ronchi D, Faranca I, Forti P, Ravaglia G, Borderi M, Manfredi R, Volterra V. Development of acute psychotic disorders and HIV-1 infection. Int J Psychiatry Med. 2000; 30(2):173-83. doi: 10.2190/PLGX-N48F-RBHJ-UF8K. PMID: 11001280.
28. Alciati A, Fusi A, D'Arminio Monforte A, Coen M, Ferri A, Mellado C. New-onset delusions and hallucinations in patients infected with HIV. J Psychiatry Neurosci. 2001 May; 26(3):229-34. PMID: 11394192; PMCID: PMC1408305.
29. Sewell D D. Schizophrenia and HIV. Schizophr Bull. 1996; 22(3):465-73. doi: 10.1093/schbul/22.3.465. PMID: 8873297.
30. Dudakov J A, Hanash A M, Jenq R R, Young L F, Ghosh A, Singer N V, West M L, Smith O M, Holland A M, Tsai J J, Boyd R L, van den Brink M R. Interleukin-22 drives endogenous thymic regeneration in mice. Science. 2012 Apr. 6; 336(6077):91-5. doi: 10.1126/science.1218004. Epub 2012 Mar. 1. PMID: 22383805
31. Keir M, Yi Y, Lu T, Ghilardi N. The role of IL-22 in intestinal health and disease. J Exp Med. 2020 Feb. 13; 217(3):e20192195. doi: 10.1084/jem.20192195.
32. Arshad T, Mansur F, Palek R, Manzoor S, Liska V. A Double Edged Sword Role of Interleukin-22 in Wound Healing and Tissue Regeneration. Front Immunol. 2020 Sep. 17; 11:2148. doi: 10.3389/fimmu.2020.02148.
33. Ezeonwumelu I J, Garcia-Vidal E, Ballana E. JAK-STAT Pathway: A Novel Target to Tackle Viral Infections. Viruses. 2021 Nov. 27; 13(12):2379. doi: 10.3390/v13122379. PMID: 34960648; PMCID: PMC8704679.
34. Perusina Lanfranca M, Lin Y, Fang J, Zou W, Frankel T. Biological and pathological activities of interleukin-22. J Mol Med (Berl). 2016 May; 94(5):523-34. doi: 10.1007/s00109-016-1391-6.
35. Fu G, Zhang W, Dai J, Liu J, Li F, Wu D, Xiao Y, Shah C, Sweeney J A, Wu M, Lui S. Increased Peripheral Interleukin 10 Relate to White Matter Integrity in Schizophrenia. Front Neurosci. 2019 Feb. 7; 13:52. doi: 10.3389/fnins.2019.00052.
36. Kapelski P, Skibinska M, Maciukiewicz M, Pawlak J, Zaremba D, Twarowska-Hauser J. Family-based association study of interleukin 10 (IL10) and interleukin 10 receptor alpha (IL10RA) functional polymorphisms in schizophrenia in Polish population. J Neuroimmunol. 2016 Aug. 15; 297:92-7. doi: 10.1016/j.jneuroim.2016.05.010.
37. Mattapallil M J, Kielczewski J L, Zárate-Blades C R, St Leger A J, Raychaudhuri K, Silver P B, Jittayasothorn Y, Chan C C, Caspi R R. Interleukin 22 ameliorates neuropathology and protects from central nervous system autoimmunity. J Autoimmun. 2019 August; 102:65-76. doi: 10.1016/j.jaut.2019.04.017. Epub 2019 May 9. PMID: 31080013; PMCID: PMC6667188
38. Rachel Caspi, Mary Mattapallil, Rachael Rigden, Carlos Zarate-Blades, Phyllis Silver, Dror Luger, Chi Chao Chan; Neuroprotective effects of IL-22 during CNS inflammation (CCR4P.203). J Immunol 1 May 2015; 194 (1_Supplement): 118.3.
39. Subbanna M, Shivakumar V, Talukdar P M, Narayanaswamy J C, Venugopal D, Berk M, Varambally S, Venkatasubramanian G, Debnath M. Role of IL-6/RORC/IL-22 axis in driving Th17 pathway mediated immunopathogenesis of schizophrenia. Cytokine. 2018 November; 111:112-118. doi: 10.1016/j.cyto.2018.08.016.
40. Barthelemy A, Sencio V, Soulard D, Deruyter L, Faveeuw C, Le Goffic R, Trottein F. Interleukin-22 Immunotherapy during Severe Influenza Enhances Lung Tissue Integrity and Reduces Secondary Bacterial Systemic Invasion. Infect Immun. 2018 Jun. 21; 86(7):e00706-17. doi: 10.1128/IAI.00706-17.
41. Albayrak N, Orte Cano C, Karimi S, Dogahe D, Van Praet A, Godefroid A, Del Marmol V, Grimaldi D, Bondue B, Van Vooren J P, Mascart F, Corbière V. Distinct Expression Patterns of Interleukin-22 Receptor 1 on Blood Hematopoietic Cells in SARS-COV-2 Infection. Front Immunol. 2022 Mar. 29; 13:769839. doi: 10.3389/fimmu.2022.769839.
42. Li L J, Gong C, Zhao M H, Feng B S. Role of interleukin-22 in inflammatory bowel disease. World J Gastroenterol. 2014 Dec. 28; 20(48):18177-88. doi: 10.3748/wjg.v20.i48.18177
43. Cella M, Fuchs A, Vermi W, Facchetti F, Otero K, Lennerz J K, Doherty J M, Mills J C, Colonna M. A human natural killer cell subset provides an innate source of IL-22 for mucosal immunity. Colonna M. Interleukin-22-producing natural killer cells and lymphoid tissue inducer-like cells in mucosal immunity. Immunity. 2009 Jul. 17; 31(1):15-23. doi: 10.1016/j.immuni.2009.06.008. Nature. 2009 Feb. 5; 457(7230):722-5. doi: 10.1038/nature07537. Epub 2008 Nov. 2.
44. Colonna M. Interleukin-22-producing natural killer cells and lymphoid tissue inducer-like cells in mucosal immunity. Immunity. 2009 Jul. 17; 31(1):15-23. doi: 10.1016/j.immuni.2009.06.008. PMID: 19604490.
45. Kumar P, Thakar M S, Ouyang W, Malarkannan S. IL-22 from conventional NK cells is epithelial regenerative and inflammation protective during influenza infection. Mucosal Immunol. 2013 January; 6(1):69-82. doi: 10.1038/mi.2012.49.
46. Zaiatz Bittencourt V, Jones F, Tosetto M, Doherty G A, Ryan E J. Dysregulation of Metabolic Pathways in Circulating Natural Killer Cells Isolated from Inflammatory Bowel Disease Patients. J Crohns Colitis. 2021 Aug. 2; 15(8):1316-1325. doi: 10.1093/ecco-jcc/jjab014. PMID: 33460436;
47. Fernandez-Egea E, Vertes P E, Flint S M, Turner L, Mustafa S, Hatton A, Smith K G, Lyons P A, Bullmore E T. Peripheral Immune Cell Populations Associated with Cognitive Deficits and Negative Symptoms of Treatment-Resistant Schizophrenia. PLOS One. 2016 May 31; 11(5):e0155631.
48. arantino N, Leboyer M, Bouleau A, Hamdani N, Richard J R, Boukouaci W, Ching-Lien W, Godin O, Bengoufa D, Le Corvoisier P, Barau C, Ledudal K, Debré P, Tamouza R, Vieillard V. Natural killer cells in first-episode psychosis: an innate immune signature? Mol Psychiatry. 2021 September; 26(9):5297-5306. doi: 10.1038/s41380-020-01008-7.
49. Steel A W, Mela C M, Lindsay J O, Gazzard B G, Goodier M R. Increased proportion of CD16(+) NK cells in the colonic lamina propria of inflammatory bowel disease patients, but not after azathioprine treatment. Aliment Pharmacol Ther. 2011 January; 33(1):115-26. doi: 10.1111/j.1365-2036.2010.04499.x.
50. Sedgwick A J, Ghazanfari N, Constantinescu P, Mantamadiotis T, Barrow A D. The Role of NK Cells and Innate Lymphoid Cells in Brain Cancer. Front Immunol. 2020 Jul. 31; 11:1549. doi: 10.3389/fimmu.2020.01549.
51. Jin W N, Shi K, He W, Sun J H, Van Kaer L, Shi F D, Liu Q. Neuroblast senescence in the aged brain augments natural killer cell cytotoxicity leading to impaired neurogenesis and cognition. Nat Neurosci. 2021 January; 24(1):61-73. doi: 10.1038/s41593-020-00745-w.
52. Vivier E, Artis D, Colonna M, Diefenbach A, Di Santo J P, Eberl G, et al. Innate Lymphoid Cells: 10 Years On. Cell. 2018 Aug. 23; 174(5):1054-1066. doi: 10.1016/j.cell.2018.07.017.
53. Artis, D., Spits, H. The biology of innate lymphoid cells. Nature 517, 293-301 (2015), doi: 10.1038/nature14189
54. Fan H, Wang A, Wang Y, Sun Y, Han J, Chen W, Wang S, Wu Y, Lu Y. Innate Lymphoid Cells: Regulators of Gut Barrier Function and Immune Homeostasis. J Immunol Res. 2019 Dec. 20; 2019:2525984. doi: 10.1155/2019/2525984.
55. Borovcanin M M, Minic Janicijevic S, Jovanovic I P, Gajovic N M, Jurisevic M M, Arsenijevic N N. Type 17 Immune Response Facilitates Progression of Inflammation and Correlates with Cognition in Stable Schizophrenia. Diagnostics (Basel). 2020 Nov. 10; 10(11):926. doi: 10.3390/diagnostics10110926.
56. Barichello T. The role of innate lymphoid cells (ILCs) in mental health. Discov Ment Health. 2022; 2(1):2. doi: 10.1007/s44192-022-00006-1. Epub 2022 Feb. 7. PMID: 35224555; PMCID: PMC8855986.
57. Yeung, S. S H., Ho, YS. & Chang, R. C C. The role of meningeal populations of type II innate lymphoid cells in modulating neuroinflammation in neurodegenerative diseases. Exp Mol Med 53, 1251-1267 (2021). https://doi.org/10.1038/s12276-021-00660-5
58. Uchiyama, K., Takagi, T., Mizushima, K. et al. Increased mucosal IL-12 expression is associated with relapse of ulcerative colitis. BMC Gastroenterol 21, 122 (2021). https://doi.org/10.1186/s12876-021-01709-5
59. Langer V, Vivi E, Regensburger D, Winkler T H, Waldner M J, Rath T, Schmid B, Skottke L, Lee S, Jeon N L, Wohlfahrt T, Kramer V, Tripal P, Schumann M, Kersting S, Handtrack C, Geppert C I, Suchowski K, Adams R H, Becker C, Ramming A, Naschberger E, Britzen-Laurent N, Sturzl M. IFN-γ drives inflammatory bowel disease pathogenesis through VE-cadherin-directed vascular barrier disruption. J Clin Invest. 2019 Nov. 1; 129(11):4691-4707. doi: 10.1172/JCI124884.
60. Kato T, Monji A, Hashioka S, Kanba S. Risperidone significantly inhibits interferon-gamma-induced microglial activation in vitro. Schizophr Res. 2007 May; 92(1-3):108-15. doi: 10.1016/j.schres.2007.01.019.
61. Wilson K E, Demyanovich H, Rubin L H, Wehring H J, Kilday C, Kelly D L. Relationship of Interferon-γ to Cognitive Function in Midlife Women with Schizophrenia. Psychiatr Q. 2018 December; 89(4):937-946. doi: 10.1007/s11126-018-9591-6
62. Arolt V, Weitzsch C, Wilke I, Nolte A, Pinnow M, Rothermundt M, Kirchner H. Production of interferon-gamma in families with multiple occurrence of schizophrenia. Psychiatry Res. 1997 Feb. 7; 66(2-3):145-52. doi: 10.1016/s0165-1781(96)03023-5
63. Mount M P, Lira A, Grimes D, Smith P D, Faucher S, Slack R, Anisman H, Hayley S, Park D S. Involvement of interferon-gamma in microglial-mediated loss of dopaminergic neurons. J Neurosci. 2007 Mar. 21; 27(12):3328-37. doi: 10.1523/JNEUROSCI.5321-06.2007.
64. Pennino D, Bhavsar P K, Effner R, Avitabile S, Venn P, Quaranta M, Marzaioli V, Cifuentes L, Durham S R, Cavani A, Eyerich K, Chung K F, Schmidt-Weber C B, Eyerich S. IL-22 suppresses IFN-γ-mediated lung inflammation in asthmatic patients. J Allergy Clin Immunol. 2013 February; 131(2):562-70. doi: 10.1016/j.jaci.2012.09.036.
65. Natah S S, Mouihate A, Pittman Q J, Sharkey K A. Disruption of the blood-brain barrier during TNBS colitis. Neurogastroenterol Motil. 2005 June; 17(3):433-46. doi: 10.1111/j.1365-2982.2005.00654.x. PMID: 15916631.
66. Chen B Y, Hsu C C, Chen Y Z, Lin J J, Tseng H H, Jang F L, Chen P S, Chen W N, Chen C S, Lin S H. Profiling antibody signature of schizophrenia by Escherichia coli proteome microarrays. Brain Behav Immun. 2022 November; 106:11-20. doi: 10.1016/j.bbi.2022.07.162.
67. Secher T, Samba-Louaka A, Oswald E, Nougayrède JP. Escherichia coli producing colibactin triggers premature and transmissible senescence in mammalian cells. PLOS One. 2013 Oct. 8; 8(10):e77157. doi: 10.1371/journal.pone.0077157.
68. Papanastasiou E, Gaughran F, Smith S. Schizophrenia as segmental progeria. J R Soc Med. 2011 November; 104(11):475-84. doi: 10.1258/jrsm.2011.110051.
69. Mizuno Y, Muraoka M, Shimabukuro K, Toda K, Miyagawa K, Yoshimatsu H, Tsuchiya M. Inflammatory bowel diseases and thymus disorder: reactivity of thymocytes with monoclonal antibodies. Bull Tokyo Dent Coll. 1990 May; 31(2):137-41. PMID: 2131166
70. Watanabe M, Funahashi T, Suzuki T, Nomura S, Nakazawa T, Noguchi T, Tsukada Y. Antithymic antibodies in schizophrenic sera. Biol Psychiatry. 1982 June; 17(6):699-710. PMID: 6125218.
71. Pan B, Wang D, Li L, Shang L, Xia F, Zhang F, Zhang Y, Gale R P, Xu M, Li Z, Xu K. IL-22 Accelerates Thymus Regeneration via Stat3/Mcl-1 and Decreases Chronic Graft-versus-Host Disease in Mice after Allotransplants. Biol Blood Marrow Transplant. 2019 October; 25(10):1911-1919. doi: 10.1016/j.bbmt.2019.06.002.
72. Falk W. A ticket to the gut for thymic T cells. Gut. 2006 July; 55(7):910-2. doi: 10.1136/gut.2005.087288. PMID: 16766746; PMCID: PMC1856347.
73. Shang L, Duah M, Xu Y, Liang Y, Wang D, Xia F, Li L, Sun Z, Yan Z, Xu K, Pan B. Dynamic of plasma IL-22 level is an indicator of thymic output after allogeneic hematopoietic cell transplantation. Life Sci. 2021 Jan. 15; 265:118849. doi: 10.1016/j.lfs.2020.118849.
74. Li Y, Wang J, Li Y, Wu H, Zhao S, Yu Q. Protecting intestinal epithelial cells against deoxynivalenol and E. coli damage by recombinant porcine IL-22. Vet Microbiol. 2019 April; 231:154-159. doi: 10.1016/j.vetmic.2019.02.027.
75. Aujla S J, Chan Y R, Zheng M, Fei M, Askew D J, Pociask D A, Reinhart T A, McAllister F, Edeal J, Gaus K, Husain S, Kreindler J L, Dubin P J, Pilewski J M, Myerburg M M, Mason C A, Iwakura Y, Kolls J K. IL-22 mediates mucosal host defense against Gram-negative bacterial pneumonia. Nat Med. 2008 March; 14(3):275-81. doi: 10.1038/nm1710.
76. Le P T, Pearce M M, Zhang S, Campbell E M, Fok C S, Mueller E R, Brincat C A, Wolfe A J, Brubaker L. IL22 regulates human urothelial cell sensory and innate functions through modulation of the acetylcholine response, immunoregulatory cytokines and antimicrobial peptides: assessment of an in vitro model. PLOS One. 2014 Oct. 29; 9(10):e111375. doi: 10.1371/journal.pone.0111375.
77. Ingersoll M A, Starkey M R. Interleukin-22 in urinary tract disease—new experimental directions. Clin Transl Immunology. 2020 Jun. 7; 9(6):e1143. doi: 10.1002/cti2.1143.
78. Ronald A. The etiology of urinary tract infection: traditional and emerging pathogens. Am J Med. 2002 Jul. 8; 113 Suppl 1A:14S-19S. doi: 10.1016/s0002-9343(02)01055-0
79. Rudzki L, Szulc A. “Immune Gate” of Psychopathology—The Role of Gut Derived Immune Activation in Major Psychiatric Disorders. Front Psychiatry. 2018 May 29; 9:205. doi: 10.3389/fpsyt.2018.00205
80. Maes M, Vojdani A, Geffard M, Moreira E G, Barbosa D S, Michelin A P, Semeão L O, Sirivichayakul S, Kanchanatawan B. Schizophrenia phenomenology comprises a bifactorial general severity and a single-group factor, which are differently associated with neurotoxic immune and immune-regulatory pathways. Biomol Concepts. 2019 Nov. 17; 10(1):209-225. doi: 10.1515/bmc-2019-0023.
81. Tang K Y, Lickliter J, Huang Z H, Xian Z S, Chen H Y, Huang C, Xiao C, Wang Y P, Tan Y, Xu L F, Huang Y L, Yan X Q. Safety, pharmacokinetics, and biomarkers of F-652, a recombinant human interleukin-22 dimer, in healthy subjects. Cell Mol Immunol. 2019 May; 16(5):473-482. doi: 10.1038/s41423-018-0029-8.
82. Arab J P, Sehrawat T S, Simonetto D A, Verma V K, Feng D, Tang T, Dreyer K, Yan X, Daley W L, Sanyal A, Chalasani N, Radaeva S, Yang L, Vargas H, Ibacache M, Gao B, Gores G J, Malhi H, Kamath P S, Shah V H. An Open-Label, Dose-Escalation Study to Assess the Safety and Efficacy of IL-22 Agonist F-652 in Patients With Alcohol-associated Hepatitis. Hepatology. 2020 August; 72(2):441-453. doi: 10.1002/hep.31046.
83. Dempsey, L. Antimicrobial IL-22. Nat Immunol 18, 373 (2017). https://doi.org/10.1038/ni.3722
84. Das S, St Croix C, Good M, Chen J, Zhao J, Hu S, Ross M, Myerburg M M, Pilewski J M, Williams J, Wenzel S E, Kolls J K, Ray A, Ray P. Interleukin-22 Inhibits Respiratory Syncytial Virus Production by Blocking Virus-Mediated Subversion of Cellular Autophagy. iScience. 2020 Jul. 24; 23(7):101256. doi: 10.1016/j.isci.2020.101256.
85. Shao, L., Xiong, X., Zhang, Y. et al. IL-22 ameliorates LPS-induced acute liver injury by autophagy activation through ATF4-ATG7 signaling. Cell Death Dis 11, 970 (2020). https://doi.org/10.1038/s41419-020-03176-4
86. Merenlender-Wagner, A., Malishkevich, A., Shemer, Z. et al. Autophagy has a key role in the pathophysiology of schizophrenia. Mol Psychiatry 20, 126-132 (2015). https://doi.org/10.1038/mp.2013.174
87. lida T, Onodera K, Nakase H. Role of autophagy in the pathogenesis of inflammatory bowel disease. World J Gastroenterol. 2017 Mar. 21; 23(11): 1944-1953. doi: 10.3748/wjg.v23.i11.1944.
88. Mo R, Lai R, Lu J, Zhuang Y, Zhou T, Jiang S, Ren P, Li Z, Cao Z, Liu Y, Chen L, Xiong L, Wang P, Wang H, Cai W, Xiang X, Bao S, Xie Q. Enhanced autophagy contributes to protective effects of IL-22 against acetaminophen-induced liver injury. Theranostics. 2018 Jul. 30; 8(15):4170-4180. doi: 10.7150/thno.25798.
89. Kim S H, Park S, Yu H S, Ko K H, Park H G, Kim Y S. The antipsychotic agent clozapine induces autophagy via the AMPK-ULK1-Beclin1 signaling pathway in the rat frontal cortex. Prog Neuropsychopharmacol Biol Psychiatry. 2018 Feb. 2; 81:96-104. doi: 10.1016/j.pnpbp.2017.10.012.
90. Kim Y K, Suh I B, Kim H, Han C S, Lim C S, Choi S H, Licinio J. The plasma levels of interleukin-12 in schizophrenia, major depression, and bipolar mania: effects of psychotropic drugs. Mol Psychiatry. 2002; 7(10):1107-14. doi: 10.1038/sj.mp.4001084. PMID: 12476326.
91. Singh R K, Dai Y, Staudinger J L, Muma N A. Activation of the JAK-STAT pathway is necessary for desensitization of 5-HT2A receptor-stimulated phospholipase C signalling by olanzapine, clozapine and MDL 100907. Int J Neuropsychopharmacol. 2009 June; 12(5):651-65. doi: 10.1017/S1461145708009590.
92. He J, Kong J, Tan Q R, Li X M. Neuroprotective effect of atypical antipsychotics in cognitive and non-cognitive behavioral impairment in animal models. Cell Adh Migr. 2009 January-March; 3(1):129-37. doi: 10.4161/cam.3.1.7401. Epub 2009 Jan. 13. PMID: 19372744; PMCID: PMC2675159.
93. Kato T, Mizoguchi Y, Monji A, Horikawa H, Suzuki S O, Seki Y, Iwaki T, Hashioka S, Kanba S. Inhibitory effects of aripiprazole on interferon-gamma-induced microglial activation via intracellular Ca2+ regulation in vitro. J Neurochem. 2008 July; 106(2):815-25. doi: 10.1111/j.1471-4159.2008.05435.x
94. Vucicevic L, Misirkic-Marjanovic M, Harhaji-Trajkovic L, Maric N, Trajkovic V. Mechanisms and therapeutic significance of autophagy modulation by antipsychotic drugs. Cell Stress. 2018 Oct. 25; 2(11):282-291. doi: 10.15698/cst2018.11.161. PMID: 31225453; PMCID: PMC6551804
95. Girgis R R, Lieberman J A. Anti-viral properties of antipsychotic medications in the time of COVID-19. Psychiatry Res. 2021 January; 295:113626. doi: 10.1016/j.psychres.2020.113626.
96. Nehme H, Saulnier P, Ramadan A A, Cassisa V, Guillet C, Eveillard M, Umerska A. Antibacterial activity of antipsychotic agents, their association with lipid nanocapsules and its impact on the properties of the nanocarriers and on antibacterial activity. PLOS One. 2018 Jan. 3; 13(1):e0189950. doi: 10.1371/journal.pone.0189950
97. Nagem R A, Ferreira Junior J R, Dumoutier L, Renauld J C, Polikarpov I. Interleukin-22 and its crystal structure. Vitam. Horm. 2006; 74:77-103. doi: 10.1016/S0083-6729(06)74004-3
98. de Oliveira Neto M, et al. Interleukin-22 forms dimers that are recognized by two interleukin-22R1 receptor chains. Biophys. J. 2008; 94:1754-1765. doi: 10.1529/biophysj.107.112664.
100. Lee D W, Zhong S, Pai R, Rae J, Sukumaran S, Stefanich E G, Lutman J, Doudement E, Wang X, Harder B, Lekkerkerker A, Herman A, Ouyang W, Danilenko D M. Nonclinical safety assessment of a human interleukin-22FC IG fusion protein demonstrates in vitro to in vivo and cross-species translatability. Pharmacol Res Perspect. 2018 Nov. 15; 6(6):e00434. doi: 10.1002/prp2.434. PMID: 30464842; PMCID: PMC6238097.
101. Yu, C., Ling, Q., Jiao, J. et al. Interleukin-22 protects from endotoxemia by inducing suppressive F4/80+Ly6GhiLy6Chi cells population. BMC Immunol 23, 45 (2022). https://doi.org/10.1186/s12865-022-00511-6
102. Stefanich E G, Rae J, Sukumaran S, Lutman J, Lekkerkerker A, Ouyang W, Wang X, Lee D, Danilenko D M, Diehl L, Loyet K M, Herman A. Pre-clinical and translational pharmacology of a human interleukin-22 IgG fusion protein for potential treatment of infectious or inflammatory diseases. Biochem Pharmacol. 2018 June; 152:224-235. doi: 10.1016/j.bcp.2018.03.031.
103. Rae, J.; Hackney, J.; Huang, K.; Keir, M.; Herman, A. Identification of an IL-22-Dependent Gene Signature as a Pharmacodynamic Biomarker. Int. J. Mol. Sci. 2021, 22, 8205. https://doi.org/10.3390/ijms22158205
104. Yorimitsu, T., Klionsky, D. Autophagy: molecular machinery for self-eating. Cell Death Differ 12 (Suppl 2), 1542-1552 (2005). https://doi.org/10.1038/sj.cdd.4401765
105. Hebert D N, Molinari M. In and out of the ER: protein folding, quality control, degradation, and related human diseases. Physiol Rev. 2007; 87:1377-408.
106. Hetz, C. The unfolded protein response: controlling cell fate decisions under ER stress and beyond. Nat Rev Mol Cell Biol 13, 89-102 (2012). https://doi.org/10.1038/nrm3270
107. Stroberg W, Eilertsen J, Schnell S. Information processing by endoplasmic reticulum stress sensors. J R Soc Interface. 2019 Sep. 27; 16(158):20190288. doi: 10.1098/rsif.2019.0288.
108. Rashid H O, Yadav R K, Kim H R, Chae H J. ER stress: Autophagy induction, inhibition and selection. Autophagy. 2015 Nov. 2; 11(11):1956-1977. doi: 10.1080/15548627.2015.1091141.
109. Senft D, Ronai Z A. UPR, autophagy, and mitochondria crosstalk underlies the ER stress response. Trends Biochem Sci. 2015 March; 40(3):141-8. doi: 10.1016/j.tibs.2015.01.002.
110. Matsuzawa-Ishimoto Y, Hwang S, Cadwell K. Autophagy and inflammation. Ann Rev Immunol. (2018) 36:73-101. doi: 10.1146/annurev-immunol-042617-053253
111. Bernstein H G, Keilhoff G, Dobrowolny H, Steiner J. Enhanced mitochondrial autophagy (mitophagy) in oligodendrocytes might play a role in white matter pathology in schizophrenia. Med Hypotheses. 2020 January; 134:109443. doi: 10.1016/j.mehy.2019.109443.
112. Lotan A, Luza S, Opazo C M, Ayton S, Lane D J R, Mancuso S, Pereira A, Sundram S, Weickert C S, Bousman C, Pantelis C, Everall I P, Bush A I. Perturbed iron biology in the prefrontal cortex of people with schizophrenia. Mol Psychiatry. 2023 Feb. 7. doi: 10.1038/s41380-023-01979-3.
113. Feng S, Chen J, Qu C, Yang L, Wu X, Wang S, Yang T, Liu H, Fang Y, Sun P. Identification of Ferroptosis-Related Genes in Schizophrenia Based on Bioinformatic Analysis. Genes (Basel). 2022 Nov. 20; 13(11):2168. doi: 10.3390/genes13112168. PMID: 36421842; PMCID: PMC9690569.
114. Apostolova N, Victor V M. Molecular strategies for targeting antioxidants to mitochondria: therapeutic implications. Antioxid Redox Signal. 2015 Mar. 10; 22(8):686-729. doi: 10.1089/ars.2014.5952.
115. Hwang S, Feng D, Gao B. Interleukin-22 acts as a mitochondria) protector. Theranostics. 2020 Jun. 19; 10(17):7836-7840. doi: 10.7150/thno.48022.
116. Smith C L, Arvedson T L, Cooke K S, Dickmann L J, Forte C, Li H, Merriam K L, Perry V K, Tran L, Rottman J B, Maxwell J R. IL-22 regulates iron availability in vivo through the induction of hepcidin. J Immunol. 2013 Aug. 15; 191(4):1845-55. doi: 10.4049/jimmunol.1202716
117. Forbester J L, Lees E A, Goulding D, Forrest S, Yeung A, Speak A, Clare S, Coomber E L, Mukhopadhyay S, Kraiczy J, Schreiber F, Lawley T D, Hancock R E W, Uhlig H H, Zilbauer M, Powrie F, Dougan G. Interleukin-22 promotes phagolysosomal fusion to induce protection against Salmonella enterica Typhimurium in human epithelial cells. Proc Natl Acad Sci USA. 2018 Oct. 2; 115(40):10118-10123. doi: 10.1073/pnas.1811866115.
118. Rauen U, Springer A, Weisheit D, Petrat F, Korth H G, et al. 2007. Assessment of chelatable mitochondrial iron by using mitochondrion-selective fluorescent iron indicators with different iron-binding affinities. ChemBioChem 8:341-52
119. Jhurry N D, Chakrabarti M, McCormick S P, Holmes-Hampton G P, Lindahl P A. 2012. Biophysical investigation of the ironome of human jurkat cells and mitochondria. Biochemistry 51:5276-84
120. Li, A., Gao, M., Liu, B. et al. Mitochondrial autophagy: molecular mechanisms and implications for cardiovascular disease. Cell Death Dis 13, 444 (2022). https://doi.org/10.1038/s41419-022-04906-6
121. Wang S, Kaufman R J. The impact of the unfolded protein response on human disease. J Cell Biol. 2012 Jun. 25; 197(7):857-67. doi: 10.1083/jcb.201110131.
122. Hu M, Yang S, Yang L, Cheng Y, Zhang H. Interleukin-22 Alleviated Palmitate-Induced Endoplasmic Reticulum Stress in INS-1 Cells through Activation of Autophagy. PLOS One. 2016 Jan. 19; 11(1):e0146818. doi: 10.1371/journal.pone.0146818
123. Muneer A, Shamsher Khan R M. Endoplasmic Reticulum Stress: Implications for Neuropsychiatric Disorders. Chonnam Med J. 2019 January; 55(1):8-19. doi: 10.4068/cmj.2019.55.1.8.
124. Jóźwiak-Bębenista M, Sokołowska P, Siatkowska M, Panek C A, Komorowski P, Kowalczyk E, Wiktorowska-Owczarek A. The Importance of Endoplasmic Reticulum Stress as a Novel Antidepressant Drug Target and Its Potential Impact on CNS Disorders. Pharmaceutics. 2022 Apr. 12; 14(4):846. doi: 10.3390/pharmaceutics 14040846.
125. Hayashi T, Su T P. Sigma-1 receptor chaperones at the ER-mitochondrion interface regulate Ca(2+) signaling and cell survival. Cell. 2007 Nov. 2; 131(3):596-610. doi: 10.1016/j.cell.2007.08.036.
126. Kim P, Scott M R, Meador-Woodruff J H. Dysregulation of the unfolded protein response (UPR) in the dorsolateral prefrontal cortex in elderly patients with schizophrenia. Mol Psychiatry. 2021 April; 26(4):1321-1331. doi: 10.1038/s41380-019-0537-7.
127. Matteoni S, Matarrese P, Ascione B, Ricci-Vitiani L, Pallini R, Villani V, Pace A, Paggi M G, Abbruzzese C. Chlorpromazine induces cytotoxic autophagy in glioblastoma cells via endoplasmic reticulum stress and unfolded protein response. J Exp Clin Cancer Res. 2021 Nov. 5; 40(1):347. doi: 10.1186/s13046-021-02144-w.
128. Mo R, Lai R, Lu J, Zhuang Y, Zhou T, Jiang S, Ren P, Li Z, Cao Z, Liu Y, Chen L, Xiong L, Wang P, Wang H, Cai W, Xiang X, Bao S, Xie Q. Enhanced autophagy contributes to protective effects of IL-22 against acetaminophen-induced liver injury. Theranostics. 2018 Jul. 30; 8(15):4170-4180. doi: 10.7150/thno.25798.
129. Mizushima N. A brief history of autophagy from cell biology to physiology and disease [Internet]. Nat Cell Bio. 2018; 20:521-527. Available from: http://www.nature.com/articles/s41556-018-0092-5
130. Nguyen H T T, Lapaquette P, Bringer M-A, et al. Autophagy and crohn's disease. J Innate Immun. [Internet] 2013; 5:434-443. Available from: https://www.karger.com/Article/FullText/345129
131. Glal D, Sudhakar J N, Lu H H, Liu M C, Chiang H Y, Liu Y C, Cheng C F, Shui J W. ATF3 Sustains IL-22-Induced STAT3 Phosphorylation to Maintain Mucosal Immunity Through Inhibiting Phosphatases. Front Immunol. 2018 Nov. 5; 9:2522. doi: 10.3389/fimmu.2018.02522.
132. Shao L, Xiong X, Zhang Y, Miao H, Ren Y, Tang X, Song J, Wang C. IL-22 ameliorates LPS-induced acute liver injury by autophagy activation through ATF4-ATG7 signaling. Cell Death Dis. 2020 Nov. 11; 11(11):970. doi: 10.1038/s41419-020-03176-4.
133. Lee J., Giordano S., Zhang J. Autophagy, mitochondria and oxidative stress: Cross¬talk and redox signalling. Biochem. J. 2012; 441:523-540. doi:
134. 10.1042/BJ20111451.
135. Levonen A. L., Hill B. G., Kansanen E., Zhang J., Darley-Usmar V. M. Redox regulation of antioxidants, autophagy, and the response to stress: Implications for electrophile therapeutics. Free Radic. Biol. Med. 2014; 71:196-207. doi: 10.1016/j.freerad biomed 0.2014.03.025.
136. Hu M, Yang S, Yang L, Cheng Y, Zhang H. Interleukin-22 Alleviated Palmitate-Induced Endoplasmic Reticulum Stress in INS-1 Cells through Activation of Autophagy. PLOS One. 2016 Jan. 19; 11(1):e0146818. doi: 10.1371/journal.pone.0146818.
137. Ye J, Wang Y, Xu Y, Wang Z, Liu L, Wang M, Ye D, Zhang J, Yang Z, Lin Y, Ji C I, Wan J. Interleukin-22 deficiency alleviates doxorubicin-induced oxidative stress and cardiac injury via the p38 MAPKimacrophage/Fizz3 axis in mice. Redox Biol. 2020 September; 36:101636. doi: 10.1016/j.redox.2020.101636.
138. Ji C, Zhang Z, Li Z, She X, Wang X, Li B, Xu X, Song D, Zhang D. Mitochondria-Associated Endoplasmic Reticulum Membranes: Inextricably Linked with Autophagy Process. Oxid Med Cell Longev. 2022 Aug. 23; 2022:7086807. doi: 10.1155/2022/7086807.
139. Park S J, Lee S B, Suh Y, Kim S J, Lee N, Hong J H, Park C, Woo Y, Ishizuka K, Kim J H, Berggren P O, Sawa A, Park S K. DISC1 Modulates Neuronal Stress Responses by Gate-Keeping ER-Mitochondria Ca2+ Transfer through the MAM. Cell Rep. 2017 Dec. 5; 21(10):2748-2759. doi: 10.1016/j.celrep.2017.11.043.
140. Machado A K, Pan A Y, da Silva T M, Duong A, Andreazza A C. Upstream Pathways Controlling Mitochondrial Function in Major Psychosis: A Focus on Bipolar Disorder. Can J Psychiatry. 2016 August; 61(8):446-56. doi: 10.1177/0706743716648297.
141. Ho G T, Theiss A L. Mitochondria and Inflammatory Bowel Diseases: Toward a Stratified Therapeutic Intervention. Annu Rev Physiol. 2022 Feb. 10; 84:435-459. doi: 10.1146/annurev-physiol-060821-083306.
142. Thoudam T, Jeon J H, Ha C M, Lee I K. Role of Mitochondria-Associated Endoplasmic Reticulum Membrane in Inflammation-Mediated Metabolic Diseases. Mediators Inflamm. 2016; 2016:1851420. doi: 10.1155/2016/1851420.
143. Xu M, Guo Y, Cheng J, Xue K, Yang M, Song X, Feng Y, Cheng J. Brain iron assessment in patients with First-episode schizophrenia using quantitative susceptibility mapping. Neuroimage Clin. 2021; 31:102736. doi: 10.1016/j.nicl.2021.102736.
144. Dietrich-Muszalska A, Kontek B. Lipid peroxidation in patients with schizophrenia. Psychiatry Clin Neurosci. 2010 October; 64(5):469-75. doi: 10.1111/j.1440-1819.2010.02132.x. PMID: 20923426.
145. Xu Y, Alfaro-Magallanes V M, Babitt J L. Physiological and pathophysiological mechanisms of hepcidin regulation: clinical implications for iron disorders. Br J Haematol. 2021 June; 193(5):882-893. doi: 10.1111/bjh.17252.
146. Liu Y S, Huang H, Zhou S M, Tian H J, Li P. Excessive Iron Availability Caused by Disorders of Interleukin-10 and Interleukin-22 Contributes to High Altitude Polycythemia. Front Physiol. 2018 May 22; 9:548. doi: 10.3389/fphys.2018.00548.
147. Keleş Altun i, Atagün Mi, Erdoğan A, Oymak Yenilmez D, Yusifova A, Şenat A, Erel Ö. Serum hepcidin/ferroportin levels in bipolar disorder and schizophrenia. J Trace Elem Med Biol. 2021 December; 68:126843. doi: 10.1016/j.jtemb.2021.126843.
148. Galy B. Extracellular iron: a signal that can be sensed and transduced in bacteria. Trends in Biochemical Sciences 2000 DOI: 10.1016/s0968-0004(00)01705-9
149. Carole L. Smith, Tara L. Arvedson, Keegan S. Cooke, Leslie J. Dickmann, Carla Forte, Hongyan Li, Kimberly L. Merriam, V. Kristina Perry, Linh Tran, James B. Rottman, Joseph R. Maxwell; IL-22 Regulates Iron Availability In Vivo through the Induction of Hepcidin. J Immunol 15 Aug. 2013; 191 (4): 1845-1855. https://doi.org/10.4049/jimmunol.1202716
150. Hennigar S R, McClung J P. Nutritional Immunity: Starving Pathogens of Trace Minerals. Am J Lifestyle Med. 2016 Feb. 4; 10(3):170-173. doi: 10.1177/1559827616629117.
151. Sakamoto K, Kim Y G, Hara H, Kamada N, Caballero-Flores G, Tolosano E, Soares M P, Puente J L, Inohara N, Núñez G. IL-22 Controls Iron-Dependent Nutritional Immunity Against Systemic Bacterial Infections. Sci Immunol. 2017 February; 2(8): eaai8371. doi: 10.1126/sciimmunol.aai8371.
152. Ermakov E A, Melamud M M, Buneva V N, Ivanova S A. Immune System Abnormalities in Schizophrenia: An Integrative View and Translational Perspectives. Front Psychiatry. 2022 Apr. 25; 13:880568. doi: 10.3389/fpsyt.2022.880568. PMID: 35546942; PMCID: PMC9082498.
153. Delaney S, Fallon B, Alaedini A, Yolken R, Indart A, Feng T, Wang Y, Javitt D. Inflammatory biomarkers in psychosis and clinical high risk populations. Schizophr Res. 2019 April; 206:440-443. doi: 10.1016/j.schres.2018.10.017.
154. Davidovic L M, Cumic J, Dugalic S, Vicentic S, Sevarac Z, Petroianu G, Corridon P, Pantic I. Gray-Level Co-occurrence Matrix Analysis for the Detection of Discrete, Ethanol-Induced, Structural Changes in Cell Nuclei: An Artificial Intelligence Approach. Microsc Microanal. 2022 February; 28(1):265-271. doi: 10.1017/S1431927621013878.
155. Satoto B D, Utoyo I, Rulaningtyas R. Feature Extraction of Gram-Negative Bacteria Texture Using Grey Level Co-Occurrence Matrix and Scale-Invariant Feature Transform. Advances in Social Science, Education and Humanities Research, volume 410 1st International Multidisciplinary Conference on Education, Technology, and Engineering (IMCETE 2019) 10.2991/assehr.k.200303.019
156. Topalovic N, Mazic S, Nesic D, Vukovic O, Cumic J, Laketic D, Stasevic Karlicic I, Pantic I. Association between Chromatin Structural Organization of Peripheral Blood Neutrophils and Self-Perceived Mental Stress: Gray-Level Co-occurrence Matrix Analysis. Microsc Microanal. 2021 Aug. 2:1-7. doi: 10.1017/S143192762101240X.
157. Shaler, C. R., Parco, A. A., Elhenawy, W. et al. Psychological stress impairs IL22-driven protective gut mucosal immunity against colonising pathobionts. Nat Commun 12, 6664 (2021). https://doi.org/10.1038/s41467-021-26992-4
158. Ge L, Liu S, Li S, Yang J, Hu G, Xu C, Song W. Psychological stress in inflammatory bowel disease: Psychoneuroimmunological insights into bidirectional gut-brain communications. Front Immunol. 2022 Oct. 6; 13:1016578. doi: 10.3389/fimmu.2022.1016578.
159. Sasabe J, Suzuki M. Emerging Role of D-Amino Acid Metabolism in the Innate Defense. Front Microbic!. 2018 May 9; 9:933. doi: 10.3389/fmicb.2018.00933
160. Taniguchi K, Sawamura H, Ikeda Y, Tsuji A, Kitagishi Y, Matsuda S. D-Amino Acids as a Biomarker in Schizophrenia. Diseases. 2022 Jan. 31; 10(1):9. doi:10.3390/diseases10010009.
161. D'Ascenzo M., Podda M. V., Grassi C. The role of D-serine as co-agonist of NMDA receptors in the nucleus accumbens: Relevance to cocaine addiction. Front. Synaptic Neurosci. 2014; 6:16.
162. de Bartolomeis A, Vellucci L, Austin M C, De Simone G, Barone A. Rational and Translational Implications of D-Amino Acids for Treatment-Resistant Schizophrenia: From Neurobiology to the Clinics. Biomolecules. 2022 Jun. 29; 12(7):909. doi: 10.3390/biom12070909.
163. Tang H, Jensen K, Houang E, McRobb F M, Bhat S, Svensson M, Bochevarov A, Day T, Dahlgren M K, Bell J A, Frye L, Skene R J, Lewis J H, Osborne J D, Tierney J P, Gordon J A, Palomero M A, Gallati C, Chapman R S L, Jones D R, Hirst K L, Sephton M, Chauhan A, Sharpe A, Tardia P, Dechaux E A, Taylor A, Waddell R D, Valentine A, Janssens H B, Aziz O, Bloomfield D E, Ladha S, Fraser I J, Ellard J M. Discovery of a Novel Class of d-Amino Acid Oxidase Inhibitors Using the Schrödinger Computational Platform. J Med Chem. 2022 May 12; 65(9):6775-6802. doi: 10.1021/acs.jmedchem.2c00118.
164. Nagano T, Yamao S, Terachi A, Yarimizu H, Itoh H, Katasho R, Kawai K, Nakashima A, Iwasaki T, Kikkawa U, Kamada S. d-amino acid oxidase promotes cellular senescence via the production of reactive oxygen species. Life Sci Alliance. 2019 Jan. 18; 2(1):e201800045. doi: 10.26508/Isa.201800045.
165. Madeira C, Freitas M E, Vargas-Lopes C, Wolosker H, Panizzutti R. Increased brain D-amino acid oxidase (DAAO) activity in schizophrenia. Schizophr Res. 2008 April; 101(1-3):76-83. doi: 10.1016/j.schres.
166. Zelante T, lannitti R G, Cunha C, De Luca A, Giovannini G, Pieraccini G, Zecchi R, D'Angelo C, Massi-Benedetti C, Fallarino F, Carvalho A, Puccetti P, Romani L. Tryptophan catabolites from microbiota engage aryl hydrocarbon receptor and balance mucosal reactivity via interleukin-22. Immunity. 2013 Aug. 22; 39(2):372-85. doi: 10.1016/j.immuni.2013.08.003.
167. Nehmar R, Fauconnier L, Alves-Filho J, Togbe D, DeCauwer A, Bahram S, Le Bert M, Ryffel B, Georgel P. Aryl hydrocarbon receptor (Ahr)-dependent II-22 expression by type 3 innate lymphoid cells control of acute joint inflammation. J Cell Mol Med. 2021 May; 25(10):4721-4731. doi: 10.1111/jcmm.16433.
168. Kumar P, Thakar M S, Ouyang W, Malarkannan S. IL-22 from conventional NK cells is epithelial regenerative and inflammation protective during influenza infection. Mucosal Immunol. 2013; 6:69-82.
169. Dudakov J A, Hanash A M, Jenq R R, Young L F, Ghosh A, Singer N V, et al. Interleukin-22 drives endogenous thymic regeneration in mice. Science. 2012; 336:91-95.
170. Aujla S J, Kolls J K. IL-22: a critical mediator in mucosal host defense. J Mol Med (Berl) 2009; 87:451-454.
171. Yu S, Peng H R, Zhang Y K, Yin Y Q, Zhou J W. Central dopaminergic control of cell proliferation in the colonic epithelium. Neurosci Res. 2022 July; 180:72-82. doi: 10.1016/j.neures.2022.02.006.
172. Mccutcheon R A, Abi-Dargham A, Howes O D. Schizophrenia, Dopamine and the Striatum: From Biology to Symptoms. Trends Neurosci. 2019 March; 42(3):205-220. doi: 10.1016/j.tins.2018.12.004.
173. Shen L H, Liao M H, Tseng Y C. Recent advances in imaging of dopaminergic neurons for evaluation of neuropsychiatric disorders. J Biomed Biotechnol. 2012; 2012:259349. doi: 10.1155/2012/259349.
174. Moncrieff J. A critique of the dopamine hypothesis of schizophrenia and psychosis. Harv Rev Psychiatry. 2009; 17(3):214-25. doi: 10.1080/10673220902979896
175. Howes O D, Kapur S. The dopamine hypothesis of schizophrenia: version III—the final common pathway. Schizophr Bull. (2009) 35:549-62. doi: 10.1093/schbul/sbp006
176. Lehrer D S, Lorenz J. Anosognosia in schizophrenia: hidden in plain sight. Innov Clin Neurosci. 2014 May; 11(5-6):10-7.
177. Nuber-Champier, A. et al. (2022) “Monocytosis in the Acute Phase of SARS-CoV-2 Infection Predicts the Presence of Anosognosia for Cognitive Deficits in the Chronic Phase”. Research Square. doi: 10.21203/rs.3.rs-1361211/v1. https://www.researchsquare.com/article/rs-1361211/v1
178. Mazza M G, Capellazzi M, Lucchi S, Tagliabue I, Rossetti A, Clerici M. Monocyte count in schizophrenia and related disorders: a systematic review and meta-analysis. Acta Neuropsychiatr. 2020 October; 32(5):229-236. doi: 10.1017/neu.2020.12.
179. Voruz P, Cionca A, Jacot de Alcântara I, Nuber-Champier A, Allali G, Benzakour L, Thomasson M, Lalive P H, Lovblad K O, Braillard O, Nehme M, Coen M, Serratrice J, et al. Functional connectivity underlying cognitive and psychiatric symptoms in post-COVID-19 syndrome: is anosognosia a key determinant? Brain Commun. 2022 Mar. 9; 4(2):fcac057. doi: 10.1093/braincomms/fcac057
180. Alvarez-Mon M A, Gómez A M, Orozco A, Lahera G, Sosa M D, Diaz D, Auba E, Albillos A, Monserrat J, Alvarez-Mon M. Abnormal Distribution and Function of Circulating Monocytes and Enhanced Bacterial Translocation in Major Depressive Disorder. Front Psychiatry. 2019 Nov. 15; 10:812. doi: 10.3389/fpsyt.2019.00812.
181. Weber N S, Gressitt K L, Cowan D N, Niebuhr D W, Yolken R H, Severance E G. Monocyte activation detected prior to a diagnosis of schizophrenia in the US Military New Onset Psychosis Project (MNOPP). Schizophr Res. 2018 July; 197:465-469. doi: 10.1016/j.schres.2017.12.016.
182. Fani Maleki A, Rivest S. Innate Immune Cells: Monocytes, Monocyte-Derived Macrophages and Microglia as Therapeutic Targets for Alzheimer's Disease and Multiple Sclerosis. Front Cell Neurosci. 2019 Jul. 31; 13:355. doi: 10.3389/fncel.2019.00355.
183. Jenkins A K, Lewis D A, Volk D W. Altered expression of microglial markers of phagocytosis in schizophrenia. Schizophr Res. 2023 January; 251:22-29. doi: 10.1016/j.schres.2022.12.005.
184. Carson R E, Naganawa M, Toyonaga T, Koohsari S, Yang Y, Chen M K, Matuskey D, Finnema S J. Imaging of Synaptic Density in Neurodegenerative Disorders. J Nucl Med. 2022 June; 63(Suppl 1):60S-67S. doi: 10.2967/jnumed.121.263201.
185. Damasio, A. Mental self: The person within. Nature 423, 227 (2003). https://doi.org/10.1038/423227a
186. Barnsley N, McAuley J H, Mohan R, Dey A, Thomas P, Moseley G L. The rubber hand illusion increases histamine reactivity in the real arm. Curr Biol. 2011 Dec. 6; 21(23):R945-6. doi: 10.1016/j.cub.2011.10.039
187. Pradeu T. Immunology and individuality. Elife. 2019 Apr. 5; 8:e47384. doi: 10.7554/eLife.47384.
188. Goudot C, Coillard A, Villani A C, Gueguen P, Cros A, Sarkizova S, Tang-Huau T L, Bohec M, Baulande S, Hacohen N, Amigorena S, Segura E. Aryl Hydrocarbon Receptor Controls Monocyte Differentiation into Dendritic Cells versus Macrophages. Immunity. 2017 Sep. 19; 47(3):582-596.e6. doi: 10.1016/j.immuni.2017.08.016.
189. Lanis J M, Alexeev E E, Curtis V F, Kitzenberg D A, Kao D J, Battista K D, Gerich M E, Glover L E, Kominsky D J, Colgan S P. Tryptophan metabolite activation of the aryl hydrocarbon receptor regulates IL-10 receptor expression on intestinal epithelia. Mucosal Immunol. 2017 September; 10(5):1133-1144. doi: 10.1038/mi.2016.133.
190. Marquet, S., Conte, I., Poudiougou, B. et al. A Functional IL22 Polymorphism (rs2227473) Is Associated with Predisposition to Childhood Cerebral Malaria. Sci Rep 7, 41636 (2017). https://doi.org/10.1038/srep41636
191. Park H, Jin U H, Karki K, Jayaraman A, Allred C, Michelhaugh S K, Mittal S, Chapkin R S, Safe S. Dopamine is an aryl hydrocarbon receptor agonist. Biochem J. 2020 Oct. 16; 477(19):3899-3910. doi: 10.1042/BCJ20200440.
192. Fehsel K, Schwanke K, Kappel B A, Fahimi E, Meisenzahl-Lechner E, Esser C, Hemmrich K, Haarmann-Stemmann T, Kojda G, Lange-Asschenfeldt C. Activation of the aryl hydrocarbon receptor by clozapine induces preadipocyte differentiation and contributes to endothelial dysfunction. J Psychopharmacol. 2022 February; 36(2):191-201. doi: 10.1177/02698811211055811.
193. Ogura J, Miyauchi S, Shimono K, Yang S, Gonchigar S, Ganapathy V, Bhutia Y D. Carbidopa is an activator of aryl hydrocarbon receptor with potential for cancer therapy. Biochem J. 2017 Sep. 28; 474(20):3391-3402. doi: 10.1042/BCJ20170583.
194. Postal B G, Ghezzal S, Aguanno D, Andre S, Garbin K, Genser L, Brot-Laroche E, Poitou C, Soula H, Leturque A, Clement K, Carrière V. AhR activation defends gut barrier integrity against damage occurring in obesity. Mol Metab. 2020 September; 39:101007. doi: 10.1016/j.molmet.2020.101007.
195. Ifergan I, Kebir H, Bernard M, Wosik K, Dodelet-Devillers A, Cayrol R, Arbour N, Prat A. The blood-brain barrier induces differentiation of migrating monocytes into Th17-polarizing dendritic cells. Brain. 2008 March; 131 (Pt 3):785-99. doi: 10.1093/brain/awm295.
196. Li Z, Liang X, Liu W, Zhao Y, Yang H, Li W, Adamovsky O, Martyniuk C J. Elucidating mechanisms of immunotoxicity by benzotriazole ultraviolet stabilizers in zebrafish (Danio rerio): Implication of the AHR-IL17/IL22 immune pathway. Environ Pollut. 2020 July; 262:114291. doi: 10.1016/j.envpol.2020.114291.
197. Genuis S J. Toxic causes of mental illness are overlooked. Neurotoxicology. 2008 November; 29(6):1147-9. doi: 10.1016/j.neuro.2008.06.005.
198. Krüger T, Long M, Bonefeld-Jørgensen E C. Plastic components affect the activation of the aryl hydrocarbon and the androgen receptor. Toxicology. 2008 Apr. 18; 246(2-3):112-23. doi: 10.1016/j.tox.2007.12.028.
199. Shum, T.-F.; Wang, L.; Chiou, J. Impact of Plasticizer on the Intestinal Epithelial Integrity and Tissue-Repairing Ability within Cells in the Proximity of the Human Gut Microbiome. Int. J. Environ. Res. Public Health 2023, 20, 2152. https://doi.org/10.3390/ijerph20032152
200. Nagayoshi H, Kakimoto K, Takagi S, Konishi Y, Kajimura K, Matsuda T. Benzotriazole ultraviolet stabilizers show potent activities as human aryl hydrocarbon receptor ligands. Environ Sci Technol. 2015 Jan. 6; 49(1):578-87. doi: 10.1021/es503926w
201. Schirmer, E., Schuster, S. & Machnik, P. Bisphenols exert detrimental effects on neuronal signaling in mature vertebrate brains. Commun Biol 4, 465 (2021). https://doi.org/10.1038/s42003-021-01966-w
202. Brown J S Jr. Effects of bisphenol-A and other endocrine disruptors compared with abnormalities of schizophrenia: an endocrine-disruption theory of schizophrenia. Schizophr Bull. 2009 January; 35(1):256-78. doi: 10.1093/schbul/sbm147.
203. Wang Y Q, Zhang H M. Exploration of binding of bisphenol A and its analogues with calf thymus DNA by optical spectroscopic and molecular docking methods. J Photochem Photobiol B. 2015 August; 149:9-20. doi: 10.1016/j.jphotobiol.2015.04.029.
204. Padma, T. Developing countries: The outcomes paradox. Nature 508, S14-S15 (2014). https://doi.org/10.1038/508S14a
205. Vassos E, Pedersen C B, Murray R M, Collier D A, Lewis C M. Meta-analysis of the association of urbanicity with schizophrenia. Schizophr Bull. 2012 November; 38(6):1118-23. doi: 10.1093/schbul/sbs096.
206. Song Y, Slominski R M, Qayyum S, Kim T K, Janjetovic Z, Raman C, Tuckey R C, Song Y, Slominski A T. Molecular and structural basis of interactions of vitamin D3 hydroxyderivatives with aryl hydrocarbon receptor (AhR): An integrated experimental and computational study. Int J Biol Macromol. 2022 Jun. 1; 209(Pt A):1111-1123. doi: 10.1016/j.ijbiomac.2022.04.048.
207. N Okudaira, et al., Induction of long interspersed nucleotide element-1 (L1) retrotransposition by 6-formylindolo[3,2-b] carbazole (FICZ), a tryptophan photoproduct. Proc Natl Acad Sci USA 107, 18487-18492 (2010).

208. Jahangir M, Li L, Zhou J S, Lang B, Wang X P. L1 Retrotransposons: A Potential Endogenous Regulator for Schizophrenia. Front Genet. 2022 Jun. 27; 13:878508. doi: 10.3389/fgene.2022.878508.
209. Karlsson P, Smith L, Farde L, Harnryd C, Sedvall G, Wiesel F A. Lack of apparent antipsychotic effect of the D1-dopamine receptor antagonist SCH39166 in acutely ill schizophrenic patients. Psychopharmacology (Berl). 1995 October; 121(3):309-16. doi: 10.1007/BF02246068
210. Deakin B, Suckling J, Dazzan P, Joyce E, Lawrie S M, Upthegrove R, Husain N, Chaudhry I B, Dunn G, Jones P B, Lisiecka-Ford D, Lewis S, Barnes T R E, Williams S C R, Pariante C M, Knox E, Drake R J, Smallman R, Barnes N M. Minocycline for negative symptoms of schizophrenia and possible mechanistic actions: the BeneMin RCT. Southampton (UK): NIHR Journals Library; 2019 August PMID: 31465163.
211. Maes M, Kanchanatawan B, Sirivichayakul S, Carvalho A F. In Schizophrenia, Increased Plasma IgM/IgA Responses to Gut Commensal Bacteria Are Associated with Negative Symptoms, Neurocognitive
212. Al-Muhanna A S, Al-Muhanna S, Alzuhairi M A. Molecular investigation of extended-spectrum beta-lactamase genes and potential drug resistance in clinical isolates of Morganella morganii. Ann Saudi Med. 2016 May-June; 36(3):223-8. doi: 10.5144/0256-4947.2016.223. PMID: 27236395; PMCID: PMC6074545.
213. Stock I, Rahman M, Sherwood K J, Wiedemann B. Natural antimicrobial susceptibility patterns and biochemical identification of Escherichia albertii and Hafnia alvei strains. Diagn Microbiol Infect Dis. 2005 March; 51(3):151-63. doi: 10.1016/j.diagmicrobio.2004.10.008.
214. Kurlandsky L E, Fader R C. In vitro activity of minocycline against respiratory pathogens from patients with cystic fibrosis. Pediatr Pulmonol. 2000 March; 29(3):210-2. doi: 10.1002/(sici) 1099-0496(200003)29:3<210::aid-ppul9>3.0.co; 2-2. PMID: 10686042.
215. Kulkarni G V. A case report of acute pediatric bacterial meningitis due to the rare isolate, Pseudomonas putida. Neuroimmunology and Neuroinflammation. 2016; 3:215-8. http://dx.doi.org/10.20517/2347-8659.2015.55
216. Chen A, Smith K P, Whitfield B A, Zucchi P C, Lasco T M, Bias T E, Kirby J E, Hirsch E B. Activity of minocycline against Klebsiella pneumoniae carbapenemase (KPC)-producing Enterobacteriaceae clinical isolates, with comparison to doxycycline and tigecycline. Diagn Microbiol Infect Dis. 2017 August; 88(4):365-367. doi: 10.1016/j.diagmicrobio.2017.05.004
217. Campbell J H, Burdo T H, Autissier P, Bombardier J P, Westmoreland S V, Soulas C, González R G, Ratai E M, Williams K C. Minocycline inhibition of monocyte activation correlates with neuronal protection in SIV neuroAIDS. PLOS One. 2011 Apr. 6; 6(4):e18688. doi: 10.1371/journal.pone.0018688. PMID: 21494695
218. Nehme H, Saulnier P, Ramadan A A, Cassisa V, Guillet C, Eveillard M, Umerska A. Antibacterial activity of antipsychotic agents, their association with lipid nanocapsules and its impact on the properties of the nanocarriers and on antibacterial activity. PLOS One. 2018 Jan. 3; 13(1):e0189950. doi: 10.1371/journal.pone.0189950.
219. Ji C, Liu N, Tu J, Li Z, Han G, Li J, Sheng C. Drug Repurposing of Haloperidol: Discovery of New Benzocyclane Derivatives as Potent Antifungal Agents against Cryptococcosis and Candidiasis. ACS Infect Dis. 2020 May 8; 6(5):768-786. doi: 10.1021/acsinfecdis.9b00197.
220. Severance E G, Gressitt K L, Stallings C R, Katsafanas E, Schweinfurth L A, Savage C L, Adamos M B, Sweeney K M, Origoni A E, Khushalani S, Leweke F M, Dickerson F B, Yolken R H. Candida albicans exposures, sex specificity and cognitive deficits in schizophrenia and bipolar disorder. NPJ Schizophr. 2016 May 4; 2:16018. doi: 10.1038/npjschz.2016.18.
221. Marco E J, Wolkowitz O M, Vinogradov S, Poole J H, Lichtmacher J, Reus V I. Double-blind antiglucocorticoid treatment in schizophrenia and schizoaffective disorder: a pilot study. World J Biol Psychiatry. 2002 July; 3(3):156-61. doi: 10.3109/15622970209150617.
222. Hawi Z, Tong J, Dark C, Yates H, Johnson B, Bellgrove M A. The role of cadherin genes in five major psychiatric disorders: A literature update. Am J Med Genet B Neuropsychiatr Genet. 2018 March; 177(2):168-180. doi: 10.1002/ajmg.b.32592.
223. Villar C. C., et al. Mucosal tissue invasion by Candida albicans is associated with E-cadherin degradation, mediated by transcription factor Rim101p and protease Sap5p. Infect Immun. 2007; 75:2126-2135.
224. Cataldi S, Codini M, Hunot S, Légeron F P, Ferri I, Siccu P, Sidoni A, Ambesi-Impiombato F S, Beccari T, Curcio F, Albi E. e-Cadherin in 1-Methyl-4-phenyl-1,2,3,6-tetrahydropyridine-Induced Parkinson Disease. Mediators Inflamm. 2016; 2016:3937057. doi: 10.1155/2016/3937057.
225. De Luca, A., Zelante, T., D'Angelo, C. et al. IL-22 defines a novel immune pathway of antifungal resistance. Mucosal Immunol 3, 361-373 (2010). https://doi.org/10.1038/mi.2010.22
226. Nasi G, Ahmed T, Rasini E, Fenoglio D, Marino F, Filaci G, Cosentino M. Dopamine inhibits human CD8+ Treg function through D1-like dopaminergic receptors. J Neuroimmunol. 2019 Jul. 15; 332:233-241. doi: 10.1016/j.jneuroim.2019.02.007
227. Sahbaz C, Zibandey N, Kurtulmus A, Duran Y, Gokalp M, Kirpinar I, Sahin F, Guloksuz S, Akkoc T. Reduced regulatory T cells with increased proinflammatory response in patients with schizophrenia. Psychopharmacology (Berl). 2020 June; 237(6):1861-1871. doi: 10.1007/s00213-020-05504-0.
228. Dansokho C, Ait Ahmed D, Aid S, Toly-Ndour C, Chaigneau T, Calle V, Cagnard N, Holzenberger M, Piaggio E, Aucouturier P, Dorothée G. Regulatory T cells delay disease progression in Alzheimer-like pathology. Brain. 2016 April; 139(Pt 4):1237-51. doi: 10.1093/brain/awv408.
229. Wang, Y., Tong, Q., Ma, SR. et al. Oral berberine improves brain dopa/dopamine levels to ameliorate Parkinson's disease by regulating gut microbiota. Sig Transduct Target Ther 6, 77 (2021). https://doi.org/10.1038/s41392-020-00456-5
230. Dichtl S, Demetz E, Haschka D, Tymoszuk P, Petzer V, Nairz M, Seifert M, Hoffmann A, Brigo N, Wurzner R, Theurl I, Karlinsey J E, Fang F C, Weiss G. Dopamine Is a Siderophore-Like Iron Chelator That Promotes Salmonella enterica Serovar Typhimurium Virulence in Mice. mBio. 2019 Feb. 5; 10(1):e02624-18. doi: 10.1128/mBio.02624-18.
231. Smith C L, Arvedson T L, Cooke K S, Dickmann L J, Forte C, Li H, Merriam K L, Perry V K, Tran L, Rottman J B, Maxwell J R. IL-22 regulates iron availability in vivo through the induction of hepcidin. J Immunol. 2013 Aug. 15; 191(4):1845-55. doi: 10.4049/jimmunol. 1202716. Epub 2013 Jul. 8. PMID: 23836059.
232. Rácz B, Spengler G. Repurposing Antidepressants and Phenothiazine Antipsychotics as Efflux Pump Inhibitors in Cancer and Infectious Diseases. Antibiotics (Basel). 2023 Jan. 10; 12(1):137. doi: 10.3390/antibiotics12010137.
233. Howland R H. Methylene Blue: The Long and Winding Road From Stain to Brain: Part 2. J Psychosoc Nurs Ment Health Serv. 2016 Oct. 1; 54(10):21-26. doi: 10.3928/02793695-20160920-04.
234. Cao X, Aballay A. Neural Inhibition of Dopaminergic Signaling Enhances Immunity in a Cell-Non-autonomous Manner. Curr Biol. 2016 Sep. 12; 26(17):2329-34. doi: 10.1016/j.cub.2016.06.036.
235. Bartels A. L., Willemsen A. T. M., Kortekaas R., et al. Decreased blood-brain barrier P-glycoprotein function in the progression of Parkinson's disease, PSP and MSA. Journal of Neural Transmission. 2008; 115(7):1001-1009. doi: 10.1007/s00702-008-0030-y.
236. Cario E. P-glycoprotein multidrug transporter in inflammatory bowel diseases: More questions than answers. World J Gastroenterol 2017; 23(9): 1513-1520 [PMID: 28321153 DOI: 10.3748/wjg.v23.i9.1513]
237. Insel T R. Rethinking schizophrenia. Nature. 2010 Nov. 11; 468(7321):187-93. doi: 10.1038/nature09552. PMID: 21068826
238. Folsom D P, McCahill M, Bartels S J, Lindamer L A, Ganiats T G, Jeste D V. Medical comorbidity and receipt of medical care by older homeless people with schizophrenia or depression. Psychiatr Serv. 2002 November; 53(11):1456-60.
239. Huxley P, Krayer A, Poole R, Prendergast L, Aryal S, Warner R. Schizophrenia outcomes in the 21st century: A systematic review. Brain Behav. 2021 June; 11(6):e02172. doi: 10.1002/brb3.2172.
240. Sfera A, Stahl S. “A century of schizophrenia: where we came from, where we are nowand where we are going” Romanian Journal of Psychopharmacology (2012) 12, 201-217
241. Delbert G. Robinson, Margaret G. Woerner, Howard M. Delman; Pharmacological Treatments for First-Episode Schizophrenia; Schizophrenia Bulletin vol. 31 no. 3 pp. 705-722, 2005, doi:10.1093/schbul/sbi032
242. Wang C, Kang Y, Liu P, Liu W, Chen W, Hayashi T, Mizuno K, Hattori S, Fujisaki H, Ikejima T. Combined use of dasatinib and quercetin alleviates overtraining-induced deficits in learning and memory through eliminating senescent cells and reducing apoptotic cells in rat hippocampus. Behav Brain Res. 2023 Feb. 25; 440:114260. doi: 10.1016/j.bbr.2022.114260.
243. Chaib, S., Tchkonia, T. & Kirkland, J. L. Cellular senescence and senolytics: the path to the clinic. Nat Med 28, 1556-1568 (2022). https://doi.org/10.1038/s41591-022-01923-y
244. Li X, Li C, Li Y, Liu C, Liang X, Liu T, Liu Z. Sodium nitroprusside protects HFD induced gut dysfunction via activating AMPKα/SIRT1 signaling. BMC Gastroenterol. 2021 Oct. 2; 21(1):359. doi: 10.1186/s12876-021-01934-y.
245. Hallak J E, Maia-de-Oliveira J P, Abrao J, Evora P R, Zuardi A W, Crippa J A, Belmonte-de-Abreu P, Baker G B, Dursun S M. Rapid improvement of acute schizophrenia symptoms after intravenous sodium nitroprusside: a randomized, double-blind, placebo-controlled trial. JAMA Psychiatry. 2013 July; 70(7):668-76. doi: 10.1001/jamapsychiatry.2013.1292. PMID: 23699763.
246. Kirchner L., Weitzdoerfer R., Hoeger H., Url A., Schmidt P., Engelmann M., et al. (2004). Impaired cognitive performance in neuronal nitric oxide synthase knockout mice is associated with hippocampal protein derangements. Nitric Oxide 11, 316-330. doi: 10.1016/j.niox.2004.10.005
247. Schairer D O, Chouake J S, Nosanchuk J D, Friedman A J. The potential of nitric oxide releasing therapies as antimicrobial agents. Virulence. 2012 May 1; 3(3):271-9. doi: 10.4161/viru.20328.
248. Fida T T, Voordouw J, Ataeian M, Kleiner M, Okpala G, Mand J, Voordouw G. Synergy of Sodium Nitroprusside and Nitrate in Inhibiting the Activity of Sulfate Reducing Bacteria in Oil-Containing Bioreactors. Front Microbiol. 2018 May 16; 9:981. doi: 10.3389/fmicb.2018.00981.
249. Teixeira A L, Rocha N P, Zhang X. Anti-NMDAR antibodies as a new piece in schizophrenia's puzzle. Future Sci OA. 2017 Mar. 3; 3(2):FSO178. doi: 10.4155/fsoa-2017-0009.
250. Shiwaku H, Nakano Y, Kato M, Takahashi H. Detection of autoantibodies against GABAARa1 in patients with schizophrenia. Schizophr Res. 2020 February; 216:543-546. doi: 10.1016/j.schres.2019.10.007.
251. Linares R, Francés R, Gutierrez A, Juanola O. Bacterial Translocation as Inflammatory Driver in Crohn's Disease. Front Cell Dev Biol. 2021 Sep. 7; 9:703310. doi: 10.3389/fcell.2021.703310.
252. Chen B Y, Hsu C C, Chen Y Z, Lin J J, Tseng H H, Jang F L, Chen P S, Chen W N, Chen C S, Lin S H. Profiling antibody signature of schizophrenia by Escherichia coli proteome microarrays. Brain Behav Immun. 2022 November; 106:11-20. doi: 10.1016/j.bbi.2022.07.162.
253. Krøll J. E. coli antibodies in schizophrenia. Psychol Med. 1986 February; 16(1):209-11. doi: 10.1017/s0033291700002646.
254. Al-Diwani A A J, Pollak T A, Irani S R, Lennox B R. Psychosis: an autoimmune disease? Immunology. 2017 November; 152(3):388-401. doi: 10.1111/imm.12795.
255. Dagorn A, Chapalain A, Mijouin L, Hillion M, Duclairoir-Poc C, Chevalier S, Taupin L, Orange N, Feuilloley M G. Effect of GABA, a bacterial metabolite, on Pseudomonas fluorescens surface properties and cytotoxicity. Int J Mol Sci. 2013 Jun. 6; 14(6):12186-204. doi: 10.3390/ijms140612186.
256. Steiner J, Walter M, Glanz W, Sarnyai Z, Bernstein H G, Vielhaber S, Kästner A, Skalej M, Jordan W, Schiltz K, Klingbeil C, Wandinger K P, Bogerts B, Stoecker W. Increased prevalence of diverse N-methyl-D-aspartate glutamate receptor antibodies in patients with an initial diagnosis of schizophrenia: specific relevance of IgG NR1a antibodies for distinction from N-methyl-D-aspartate glutamate receptor encephalitis. JAMA Psychiatry. 2013 March; 70(3):271-8. doi: 10.1001/2013.jamapsychiatry.86.
257. Nakayama Y, Hashimoto K I, Sawada Y, Sokabe M, Kawasaki H, Martinac B. Corynebacterium glutamicum mechanosensitive channels: towards unpuzzling “glutamate efflux” for amino acid production. Biophys Rev. 2018 October; 10(5):1359-1369. doi: 10.1007/s12551-018-0452-1.
258. Zhao N, Liu C, Li N, Zhou S, Guo Y, Yang S, Liu H. Role of Interleukin-22 in ulcerative colitis. Biomed Pharmacother. 2023 March; 159:114273. doi: 10.1016/j.biopha.2023.114273.
259. Ihekweazu F D, Engevik M A, Ruan W, Shi Z, Fultz R, Engevik K A, Chang-Graham A L, Freeborn J, Park E S, Venable S, Horvath T D, Haidacher S J, Haag A M, Goodwin A, Schady D A, Hyser J M, Spinler J K, Liu Y, Versalovic J. Bacteroides ovatus Promotes IL-22 Production and Reduces Trinitrobenzene Sulfonic Acid-Driven Colonic Inflammation. Am J Pathol. 2021 April; 191(4):704-719. doi: 10.1016/j.ajpath.2021.01.009.
260. Tan H, Yu Z, Wang C, Zhang Q, Zhao J, Zhang H, Zhai Q, Chen W. Pilot Safety Evaluation of a Novel Strain of Bacteroides ovatus. Front Genet. 2018 Nov. 6; 9:539. doi: 10.3389/fgene.2018.00539.
261. Sun S, Luo L, Liang W, Yin Q, Guo J, Rush A M, Lv Z, Liang Q, Fischbach M A, Sonnenburg J L, Dodd D, Davis M M, Wang F. Bifidobacterium alters the gut microbiota and modulates the functional metabolism of T regulatory cells in the context of immune checkpoint blockade. Proc Natl Acad Sci USA. 2020 Nov. 3; 117(44):27509-27515. doi: 10.1073/pnas.1921223117.
262. Wang W, Chen L, Zhou R, Wang X, Song L, Huang S, Wang G, Xia B. Increased proportions of Bifidobacterium and the Lactobacillus group and loss of butyrate-producing bacteria in inflammatory bowel disease. J Clin Microbiol. 2014 February; 52(2):398-406. doi: 10.1128/JCM.01500-13.
263. Hüseyin Bozkurt, PROBIOTIC BIFIDOBACTERIUM AND A NEW GENERATION POSTBIOTIC: ALGINIC ACID, Inflammatory Bowel Diseases, Volume 29, Issue Supplement_1, February 2023, Page S48, https://doi.org/10.1093/ibd/izac247.091
264. Zhang Z, Wang X, Li F. An exploration of alginate oligosaccharides modulating intestinal inflammatory networks via gut microbiota. Front Microbiol. 2023 Jan. 26; 14:1072151. doi: 10.3389/fmicb.2023.1072151.
265. Ciofani M, Madar A, Galan C, Sellars M, Mace K, Pauli F, Agarwal A, Huang W, Parkhurst C N, Muratet M, Newberry K M, Meadows S, Greenfield A, Yang Y, Jain P, Kirigin F K, Birchmeier C, Wagner E F, Murphy K M, Myers R M, Bonneau R, Littman D R. A validated regulatory network for Th17 cell specification. Cell. 2012 Oct. 12; 151(2):289-303. doi: 10.1016/j.cell.2012.09.016.
266. Ventevogel M S, Sempowski G D. Thymic rejuvenation and aging. Curr Opin Immunol. 2013 August; 25(4):516-22. doi: 10.1016/j.coi.2013.06.002.
267. Malmqvist A, Schwieler L, Orhan F, Fatouros-Bergman H, Bauer M, Flyckt L, Cervenka S, Engberg G, Piehl F; Karolinska Schizophrenia Project (KaSP) consortium; Erhardt S. Increased peripheral levels of TARC/CCL17 in first episode psychosis patients. Schizophr Res. 2019 August; 210:221-227. doi: 10.1016/j.schres.2018.12.033.
268. Sorrentino S, Ascari R, Maderna E, Catania M, Ghetti B, Tagliavini F, Giaccone G, Di Fede G. Microglial Heterogeneity and Its Potential Role in Driving Phenotypic Diversity of Alzheimer's Disease. Int J Mol Sci. 2021 Mar. 9; 22(5):2780. doi: 10.3390/ijms22052780
269. Zhang Y, Ye Y, Tang X, Wang H, Tanaka T, Tian R, Yang X, Wang L, Xiao Y, Hu X, Jin Y, Pang H, Du T, Liu H, Sun L, Xiao S, Dong R, Ferrucci L, Tian Z, Zhang S. CCL17 acts as a novel therapeutic target in pathological cardiac hypertrophy and heart failure. J Exp Med. 2022 Aug. 1; 219(8):e20200418. doi: 10.1084/jem.20200418
270. Lue L F, Schmitz C T, Snyder N L, Chen K, Walker D G, Davis K J, Belden C, Caviness J N, Driver-Dunckley E, Adler C H, Sabbagh M N, Shill H A. Converging mediators from immune and trophic pathways to identify Parkinson disease dementia. Neurol Neuroimmunol Neuroinflamm. 2016 Jan. 27; 3(1):e193. doi: 10.1212/NXI.0000000000000193.
271. Taube C, Tertilt C, Gyülveszi G, Dehzad N, Kreymborg K, Schneeweiss K, Michel E, Reuter S, Renauld J C, Arnold-Schild D, Schild H, Buhl R, Becher B. IL-22 is produced by innate lymphoid cells and limits inflammation in allergic airway disease. PLOS One. 2011; 6(7):e21799. doi: 10.1371/journal.pone.0021799.
272. Akahoshi E, Yoshimura S, Ishihara-Sugano M. Over-expression of AhR (aryl hydrocarbon receptor) induces neural differentiation of Neuro2a cells: neurotoxicology study. Environ Health. 2006 Sep. 7; 5:24. doi: 10.1186/1476-069X-5-24.
273. Ahmed S, Shibazaki M, Takeuchi T, Kikuchi H. Protein kinase Ctheta activity is involved in the 2,3,7,8-tetrachlorodibenzo-p-dioxin-induced signal transduction pathway leading to apoptosis in L-MAT, a human lymphoblastic T-cell line. Febs J. 2005; 272:903-915.
275. Monteleone I, Rizzo A, Sarra M, Sica G, Sileri P, Biancone L, MacDonald T T, Pallone F, Monteleone G. Aryl hydrocarbon receptor-induced signals up-regulate IL-22 production and inhibit inflammation in the gastrointestinal tract. Gastroenterology. 2011 July; 141(1):237-48, 248.e1. doi: 10.1053/j.gastro.2011.04.007.
276. Xue J, Nguyen D T, Habtezion A. Aryl hydrocarbon receptor regulates pancreatic IL-22 production and protects mice from acute pancreatitis. Gastroenterology. 2012 December; 143(6):1670-80. doi: 10.1053/j.gastro.2012.08.051.
277. Schiering C, Vonk A, Das S, Stockinger B, Wincent E. Cytochrome P4501-inhibiting chemicals amplify aryl hydrocarbon receptor activation and IL-22 production in T helper 17 cells. Biochem Pharmacol. 2018 May; 151:47-58. doi: 10.1016/j.bcp.2018.02.031.
278. Smith S H, Jayawickreme C, Rickard D J, Nicodeme E, Bui T, Simmons C, Coquery C M, Neil J, Pryor W M, Mayhew D, Rajpal D K, Creech K, Furst S, Lee J, Wu D, Rastinejad F, Willson T M, Viviani F, Morris D C, Moore J T, Cote-Sierra J. Tapinarof Is a Natural AhR Agonist that Resolves Skin Inflammation in Mice and Humans. J Invest Dermatol. 2017 October; 137(10):2110-2119. doi:

10.1016/j.jid.2017.05.004.
279. Safe, S.; Jin, U.-h.; Park, H.; Chapkin, R. S.; Jayaraman, A. Aryl Hydrocarbon Receptor (AHR) Ligands as Selective AHR Modulators (SAhRMs). Int. J. Mol. Sci. 2020, 21, 6654. https://doi.org/10.3390/ijms21186654
280. Jin, U. H.; Park, H.; Li, X.; Davidson, L. A.; Allred, C.; Patil, B.; Jayaprakasha, G.; Orr, A. A.; Mao, L.; Chapkin, R. S.; et al. Structure-Dependent Modulation of Aryl Hydrocarbon Receptor-Mediated Activities by Flavonoids. Toxicol. Sci. 2018, 164, 205-217
281. Bartoňková I, Dvořák Z. Essential oils of culinary herbs and spices display agonist and antagonist activities at human aryl hydrocarbon receptor AhR. Food Chem Toxicol. 2018 January; 111:374-384. doi: 10.1016/j.fct.2017.11.049.
282. Goya-Jorge E, Jorge Rodríguez M E, Veitía M S, Giner R M. Plant Occurring Flavonoids as Modulators of the Aryl Hydrocarbon Receptor. Molecules. 2021 Apr. 16; 26(8):2315. doi: 10.3390/molecules26082315.
283. Khdera H A, Saad S Y, Moustapha A, Kandil F. Synthesis of new flavonoid derivatives based on 3-hydroxy-4′-dimethylamino flavone and study the activity of some of them as antifungal. Heliyon. 2022 Dec. 5; 8(12):e12062. doi: 10.1016/j.heliyon.2022.e12062.
284. Bindhu Madhavi Rayaprolu, Jonathan J. Strawser & Gopal Anyarambhatla (2018) Excipients in parenteral formulations: selection considerations and effective utilization with small molecules and biologics, Drug Development and Industrial Pharmacy, 44:10, 1565-1571, DOI: 10.1080/03639045.2018.1483392
285. Liu R, Tang W, Wang W, Xu F, Fan W, Zhang Y, Zhang C. NLRP3 Influences Cognitive Function in Schizophrenia in Han Chinese. Front Genet. 2021 Dec. 10; 12:781625. doi: 10.3389/fgene.2021.781625
286. Chen Q L, Yin H R, He Q Y, Wang Y. Targeting the NLRP3 inflammasome as new therapeutic avenue for inflammatory bowel disease. Biomed Pharmacother. 2021 June; 138:111442. doi: 10.1016/j.biopha.2021.111442. Epub 2021 Mar. 2. PMID: 33667791.
287. Maes M, Kanchanatawan B, Sirivichayakul S, Carvalho A F. In Schizophrenia, Increased Plasma IgM/IgA Responses to Gut Commensal Bacteria Are Associated with Negative Symptoms, Neurocognitive Impairments, and the Deficit Phenotype. Neurotox Res. 2019 April; 35(3):684-698. doi: 10.1007/s12640-018-9987-y.
288. Xiu M H, Yang G G, Tan Y L, Chen D C, Tan S P, Wang Z R, Yang F D, Okusaga O, Soares J C, Zhang X Y. Decreased interleukin-10 serum levels in first-episode drug-naïve schizophrenia: relationship to psychopathology. Schizophr Res. 2014 June; 156(1):9-14. doi: 10.1016/j.schres.2014.03.024
289. Sun, Y., Ma, J., Li, D. et al. Interleukin-10 inhibits interleukin-13 production and inflammasome activation of microglia in epileptic seizures. J Neuroinflammation 16, 66 (2019). doi: 10.1186/s12974-019-1452-1
290. Cheng, X., Xie, Y., Wang, A. et al. Correlation between elevated serum interleukin-1B, interleukin-16 levels and psychiatric symptoms in patients with schizophrenia at different stages. BMC Psychiatry 23, 396 (2023). doi: 10.1186/s12888-023-04896-5
291. Reisinger S N, Sideromenos S, Horvath O, Derdak S, Cicvaric A, Monje F J, Bilban M, Haring M, Glat M, Pollak D D. STAT3 in the dorsal raphe gates behavioural reactivity and regulates gene networks associated with psychopathology. Mol Psychiatry. 2021 July; 26(7):2886-2899. doi: 10.1038/s41380-020-00904-2
292. Niklison-Chirou M V, Agostini M, Amelio I, Melino G. Regulation of Adult Neurogenesis in Mammalian Brain. Int J Mol Sci. 2020 Jul. 9; 21(14):4869. doi: 10.3390/ijms21144869.
293. Coronas V, Arnault P, Jegou J F, Cousin L, Rabeony H, Clarhaut S, Harnois T, Lecron J C, Morel F. IL-22 Promotes Neural Stem Cell Self-Renewal in the Adult Brain. Stem Cells. 2023 Mar. 17; 41(3):252-259. doi: 10.1093/stmcls/sxad003. PMID: 36635952
294. Sabat R, Ouyang W, Wolk K. Therapeutic opportunities of the IL-22-IL-22R1 system. Nat. Rev. Drug. Discov. 2014; 13:21-38. doi: 10.1038/nrd4176
295. Hu L, Zhang L. Adult neural stem cells and schizophrenia. World J Stem Cells. 2022 Mar. 26; 14(3):219-230. doi: 10.4252/wjsc.v14.i3.219. PMID: 35432739; PMCID: PMC896821
296. Park O, et al. In vivo consequences of liver-specific interleukin-22 expression in mice: implications for human liver disease progression. Hepatology. 2011; 54:252-261. doi: 10.1002/hep.24339
297. Dambaeva S, Schneiderman S, Jaiswal M K, Agrawal V, Katara G K, Gilman-Sachs A, Hirsch E, Beaman K D. Interleukin 22 prevents lipopolysaccharide-induced preterm labor in mice. Biol Reprod. 2018 Mar. 1; 98(3):299-308. doi: 10.1093/biolre/iox182. PMID: 29315356; PMCID: PMC6669419
298. Eyerich S, Eyerich K, Pennino D, Carbone T, Nasorri F, Pallotta S, et al. Th22 cells represent a distinct human T cell subset involved in epidermal immunity and remodeling. J Clini Investigat. (2009) 119:3573-85 DOI: 10.1172/JCI40202
299. Trifari S, Kaplan C D, Tran E H, Crellin N K, Spits H. Identification of a human helper T cell population that has abundant production of interleukin 22 and is distinct from TH-17, TH1 and TH2 cells. Nat Immunol. 2009; 10:864-871, DOI: 10.1038/ni.1770
300. Duhen T, Geiger R, Jarrossay D, Lanzavecchia A, Sallusto F. Production of interleukin 22 but not interleukin 17 by a subset of human skin-homing memory T cells. Nature Immunol. (2009) 10:857-63, DOI: 10.1038/ni.1767.
301. Shen, J., Wang, Y., Zheng, F. et al. Aryl hydrocarbon receptor regulates IL-22 receptor expression on thymic epithelial cell and accelerates thymus regeneration. npj Regen Med 8, 64 (2023). https://doi.org/10.1038/s41536-023-00339-7
302. Jacob, A., Hartz, A. M., Potin, S. et al. Aryl hydrocarbon receptor-dependent upregulation of Cyp1b1 by TCDD and diesel exhaust particles in rat brain microvessels. Fluids Barriers CNS 8, 23 (2011). DOI: 10.1186/2045-8118-8-23
303. Fehsel K, Schwanke K, Kappel B A, Fahimi E, Meisenzahl-Lechner E, Esser C, Hemmrich K, Haarmann-Stemmann T, Kojda G, Lange-Asschenfeldt C. Activation of the aryl hydrocarbon receptor by clozapine induces preadipocyte differentiation and contributes to endothelial dysfunction. J Psychopharmacol. 2022 February; 36(2):191-201. doi: 10.1177/02698811211055811
304. Alabbas, S. Y., Begun, J., Florin, T. H. & Oancea, I. The role of IL-22 in the resolution of sterile and nonsterile inflammation. Clin. Transl. Immunol. 7, e1017 (2018), DOI: 10.1002/cti2.1017
305. Li L J, Gong C, Zhao M H, Feng B S. Role of interleukin-22 in inflammatory bowel disease. World J Gastroenterol. 2014 Dec. 28; 20(48):18177-88. doi: 10.3748/wjg.v20.i48.18177. PMID: 25561785; PMCID: PMC4277955
306. Dumoutier, L. et al., “Human interleukin-10-related T cell-derived inducible factor: Molecular cloning and functional characterization as an hepatocyte-stimulating factor,” PNAS, 2000, 97(18) 10144-10149, doi: 10.1073/pnas.170291697.
307. Dumoutier L, Louahed J, Renauld J C. Cloning and characterization of IL-10-related T cell-derived inducible factor (IL-TIF), a novel cytokine structurally related to IL-10 and inducible by IL-9. J Immunol. 2000 Feb. 15; 164(4):1814-9. doi: 10.4049/jimmunol. 164.4.1814. PMID: 10657629.
308. Wolk K, Kunz S, Asadullah K, Sabat R. Cutting edge: immune cells as sources and targets of the IL-10 family members? J Immunol. 2002 Jun. 1; 168(11):5397-402. doi: 10.4049/jimmunol. 168.11.5397. PMID: 12023331.
309. Sfera, A. et al. Microbial Translocation Disorders: Assigning an Etiology to Idiopathic Illnesses. Appl. Microbiol. 2023, 3, 212-240. Doi: 10.3390/applmicrobiol3010015
310. Foxall R, Fernandes S, Sousa A. Profile of interleukin-22 in gut mucosal health and disease. International Journal of Interferon, Cytokine and Mediator Research. 2016; 8:1-11 doi: 10.2147/IJICMR.S64856

1. A method of treating schizophrenia or psychosis, comprising the administration of a pharmaceutical formulation containing and active agent comprising IL-22 or a dimer, fusion protein or conjugate thereof to a person suffering from schizophrenia.

2. The method of treating schizophrenia or psychosis of claim 1 wherein the active agent is interleukin-22 having the sequence of SEQ ID NO: 1 (179 amino acid sequence of IL-22) or F-652 having SEQ ID NO: 2.

3. The method of claim 1 wherein the active agent is F-652.

4. The method of claim 1, wherein the active agent is administered in an injectable formulation containing at least one excipient selected from a bulking agent, a buffering agent, a antimicrobial preservative, a chelating agent, an antioxidant, a reducing agent, a solvent, and a cosolvent, or a combination thereof.

5. The method of claim 4, wherein the injection is administered subcutaneously, by intramuscular injection or intravenously.

6. The method of claim 1 wherein the dose of the active agent is 1.0 μg/kg of body weight to 200 μg/kg of body weight.

7. The method of claim 6, wherein the dose of the active agent is 5.0 μg/kg of body weight to 100 μg/kg of body weight or 25 μg/kg of body weight to 75 μg/kg of body weight or 45 μg/kg of body weight.

8. The method of claim 6, wherein the dose of the active agent is administered once per day, two times per week, once per week, once every 14 days, once every 21 days, or once per month.

9. The method of treating schizophrenia or psychosis of claim 1 wherein the active agent inhibits microbial translocation in the intestines.

10. The use of F-652 for the treatment of schizophrenia or psychosis.

11. A pharmaceutical composition for the treatment of schizophrenia or psychosis, wherein the composition comprises an interleukin-22 agonist.

12. The pharmaceutical composition of claim 11, wherein the interleukin-22 agonist is F-652.
